(19) United States
(12) Patent Application Publication
(10) Pub. No.: US 2013/0245116 A1
(43) Pub. Date: Sep. 19, 2013

(54) COMPOSITIONS AND METHODS FOR THE DISRUPTION OF MYCOBACTERIUM BIOFILM

(71) Applicants: Kuni Takayama, Madison, WI (US); Soofia Siddiqui, Middleton, WI (US); Martha M. Vestling, Madison, WI (US)

(72) Inventors: Kuni Takayama, Madison, WI (US); Soofia Siddiqui, Middleton, WI (US); Martha M. Vestling, Madison, WI (US)

(73) Assignees: U.S. Department of Veterans Affairs; Wisconsin Alumni Research Foundation

(21) Appl. No.: 13/611,834

(22) Filed: Sep. 12, 2012

Related U.S. Application Data
(60) Provisional application No. 61/535,856, filed on Sep. 16, 2011.
(60) Provisional application No. 61/542,051, filed on Sep. 30, 2011.

(51) Int. Cl. A01N 37/12; A61K 31/232; C11B 1/10

(52) U.S. Cl. CPC A01N 37/12; C11B 1/10; A61K 31/232
USPC 514/547; 554/224

(57) ABSTRACT

The present disclosure provides methods and compositions for the disruption of biofilms produced by Mycobacterium. Methods and compositions related to the prevention and treatment of Mycobacterium infections in which biofilm formation is implicated are also provided.

### CROSS-REFERENCE TO RELATED APPLICATIONS

The present application claims the benefit under 35 U.S.C. §119(e) of U.S. Provisional Application Ser. No. 61/535,856, entitled “Compositions And Methods for the Disruption of Mycobacterium Biofilm,” filed Sep. 16, 2011, and U.S. Provisional Application Ser. No. 61/542,051, filed Sep. 30, 2011, entitled “Compositions And Methods for the Disruption of Mycobacterium Biofilm,” the specification, drawings, claims and abstract of which are incorporated herein by reference in their entirety.

### BACKGROUND

Tuberculosis (TB) is the world's deadliest disease. Each year, eight million people world-wide become sick with TB and over two million of them die. One-third of the world's population is estimated to be infected with TB. Mycobacterium tuberculosis, the causative agent of TB, is clearly one of the most predominant global human pathogens. Due to long-term chemotherapeutic regimens (use of multiple drugs daily for 6-9 months), poor compliance and follow-up, multiple drug-resistant strains of M. tuberculosis have emerged. There is accordingly an urgent need to either improve the efficacy of current drug regimen or develop new drugs which are effective against TB.

Biofilms are surface-bound, sessile communities of microbial cells (as opposed to individual cells in suspension as in planktonic growth) and are thought to be involved in bacterial antibiotic resistance. Biofilms of M. avium and M. chelonae have been implicated in the extraordinary survival during starvation and resistance to antibiotics of these microorganisms. M. tuberculosis forms biofilms which contain an extra-cellular matrix rich in free mycolic acids. This biofilm can harbor drug-tolerant population of bacilli that persists despite exposure to very high levels of anti-TB drugs. This may be the reason why all current chemotherapeutic regimens for TB require long-term therapy. Preventing the formation of M. tuberculosis biofilm has the potential make drug treatment for TB more effective.

### SUMMARY

The present application relates generally to methods and compositions for the disruption of microbial biofilms, e.g., biofilms associated with Mycobacterium. In certain embodiments, the present technology relates to the disruption of biofilms produced by or comprising Mycobacterium species. Certain embodiments of the present technology relate to methods for the treatment of Mycobacterium infections, e.g., M. tuberculosis infections, through the disruption of Mycobacterium biofilms.

In some embodiments, the present application provides a structural series of lipids that is capable of disrupting the formation of mycobacterial biofilm. For example, planktonic culture of M. smegmatis produces a structural series of a lipids that can inhibit the formation of mycobacterial biofilm. These lipids are structurally related to the aglycone of certain mycobacterial mycosides.

The present lipid compounds, which in some embodiments are capable of disrupting the formation of mycobacterial biofilm, are structurally related to phthiocerol diesters isolated from Mycobacterium. Suitable examples of such compounds includes compounds having the formula:

CH₃(CH₂)ₙ—CH(OR)—CH₂—CH(OR′)—(CH₂)₄—CH(CH₃)—CH(OH)—R″

in which R″ is typically a lower alkyl group (i.e., an alkyl group having from 1 to 6 carbon atoms), such as a methyl or ethyl group; and one of R and R′ is an unsaturated fatty acyl group and the other is a polymethyl substituted fatty acyl group (such as an acyl group derived from a mycocerosic acid). The polymethyl substituted fatty acyl group may commonly be an acyl group represented by the formula (where “m” and “x” are integers):

CH₃—(CH₂)ₘ—(CH₂—CH(CH₃))ₓ—C(O)—

Examples of such polymethyl substituted fatty acyl groups include acyl groups derived from mycoserosic acids.

For example, in some embodiments, the lipid compounds have a formula:

CH₃(CH₂)ₙ—CH(OR)—CH₂—CH(OR′)—(CH₂)₄—CH(CH₃)—CH(OH)—R″

in which R″ is typically a methyl or ethyl group; one of R and R′ is a polyunsaturated fatty acyl group; and one of R and R′ is an aliphatic acyl group having a formula:

CH₃—(CH₂)ₘ—(CH₂—CH(CH₃))ₓ—C(O)—

wherein m is an integer from 8 to 22; and x is 3 or 4. The polyunsaturated fatty acyl group may be a 16-carbon polyunsaturated fatty acyl group or an 18 carbon polyunsaturated acyl group.

In some embodiments, the present lipid compounds have a formula:

CH₃(CH₂)ₙ—CH(OR)—CH₂—CH(OR′)—(CH₂)₄—CH(CH₃)—CH(OH)—R″

in which R″ is typically a methyl or ethyl group; one of R and R′ is an omega-3 fatty acyl group; and one of R and R′ is an aliphatic acyl group having a formula:

CH₃—(CH₂)ₘ—(CH₂—CH(CH₃))ₓ—C(O)—

wherein m is an integer from 8 to 22; and x is 3 or 4. The omega-3 fatty acyl group may be a polyunsaturated fatty acyl group, such as a 16 carbon omega-3 fatty acyl group or an 18 carbon omega-3 fatty acyl group (e.g., an omega-3 fatty acyl group derived from linolenic acid or linolenic acid).

In some embodiments, the polyunsaturated fatty acyl group is represented by the formula:

CH₃—CH₂—CH═CH—(CH₂)ₐ—CH═CH—(CH₂)_{b}—CH═CH—CH₂—C(O)—,

where a and b are integers and a+b=6.

In some embodiments, the omega-3 fatty acyl group is represented by the formula:

CH₃—CH₂—CH═CH—(CH₂)ₐ—CH═CH—(CH₂)_{b}—CH═CH—CH₂—C(O)—,

where a and b are integers and a+b=6.

In one aspect the present disclosure provides a method for disrupting biofilm produced by Mycobacterium comprising contacting the biofilm with an effective amount of a composition comprising the compound of any of claims 1-36. In some embodiments, the Mycobacterium is Mycobacterium tuberculosis. In some embodiments, the Mycobacterium is Mycobacterium smegmatis.

In one aspect the present disclosure provides a composition for disrupting biofilm produced by Mycobacterium comprising an effective amount of the compound of any of claims 1-36. In another aspect the present disclosure provides a method for aiding dispersal of biofilm produced by Mycobacterium comprising contacting the biofilm with an effective amount of a composition comprising the compound of any of claims 1-36. In another aspect the present disclosure provides a method for inhibiting formation of biofilm produced by Mycobacterium on a surface, comprising contacting the surface with an effective amount of a composition comprising the compound of any of claims 1-36.

In one aspect the present disclosure provides a method for treating Mycobacterium infection in a subject in need thereof, comprising administering to the subject an effective amount of a composition comprising the compound of any of claims 1-36. In some embodiments, the composition is administered in conjunction with one or more anti-Mycobacterium agents.

In one aspect the present disclosure provides a pharmaceutical composition for treating a Mycobacterium infection comprising an effective amount of the compound of any of claims 1-36 and a pharmaceutically acceptable carrier. In some embodiments, the composition is formulated for pulmonary administration. In some embodiments, the composition is formulated for administration by any one of intratracheal instillation, intratracheal delivery of liposomes, insufflation, nebulization, dry powder inhalation, aerosol inhalation, or bronchoalveolar lavage. In some embodiments, the composition comprises a propellant selected from the group consisting of hydrofluoroalkanes, chlorofluorocarbons, propane, nitrogen, or a mixture thereof. In some embodiments, the compound is formulated for controlled release.

### BRIEF DESCRIPTION OF THE FIGURES

FIG. 1 Effect of partially purified lipid from CF and CP of M. smegmatis on dispersion of M. smegmatis biofilm. Biofilms were grown in M63 medium on acetone-etched 96-well polystyrene microtiter plate for 8 days. The mature biofilms were treated with 200 μg/ml of silica gel column purified fractions dissolved in DMSO for 60 minutes. Sodium dodecyl sulfate (SDS) (2%) was used as a positive control. (*) P<0.05 versus DMSO control and (†) P>0.01 versus control.

FIG. 2 TLC of C₁₈-bonded silica gel cartridge fractions from M. smegmatis CF. (A) Lane-1, 100 μg of methanol effluent; lane-2, 10 μg of TMM. (B) Lane-3, 50 μg of M/C (4:1) effluent; lane-4, 50 μg of M/C (1:1) effluent. The biologically active bands are b-3, multiple band b-6 and the double band c-5. Silica gel plate was used with the solvent systems of CMN (85:15:1.5) for A and (90:10:1) for B.

FIG. 3 Biofilm inhibition assay of C₁₈-bonded silica gel cartridge fractions from the CF. TLC-purified samples of the cartridge fractions were assayed and the tubes were photographed on day-4. In all cases, there was no addition to tube-1 and 20 μl of CM (2:1) was added to tube-2. (A) Tube-3, 30 μg/ml of M/C (4:1) effluent/TLC-7; tube-4, 60 μg/ml of M/C (4:1) effluent/TLC-7. (B) Tube-3, 30 μg/ml of M/C (1:1) effluent/TLC-6; tube-4, 60 μg/ml of M/C (1:1) effluent/TLC-6. (C) Tube-3, 30 μg/ml of M/C (1:1) effluent/TLC-7; tube-4, 60 μg/ml of M/C (1:1) effluent/TLC-7.

FIG. 4 TLC of B1 to B3 and TLC sub-fractions of B1 from the CM-extract of M. smegmatis. (A) Lane-1, B1; lane-2, B2; lane-3, B3 at 30 μg. (B) Lane-4, B1-1; lane-5, B1-2; lane-6, B1-3 at 50 μg. Silica gel plate was used with the solvent systems of CMN (92:8:0.8) for A and (90:10:1) for B.

FIG. 5 Biofilm inhibition assay of purified CM-extract of M. smegmatis. The CM-soluble/methanol-soluble fraction of the CM-extract of M. smegmatis and its silica gel column/TLC fractions were assayed and the tubes were photographed on day-4. In all cases, there was no addition to tube-1 and 10 μl of CM (2:1) was added to tube-2. (A) Tube-3, 25 μg/ml B1; tube-4, 12.5 μg/ml B1; tube-5, 5 μg/ml B1. (B) Tube-3, 25 μg/ml B1-3; tube-4, 12.5 μg/ml B1-3.

FIG. 6. MALDI mass spectra of (A) B1 and (B) acetylated B1. B1 was acetylated with acetyl-chloride. Acetylation of a hydroxyl group increases the mass of the new products by 42 amu.

FIG. 7. Positive ion MALDI mass spectrum of α-MAME from M. tuberculosis H37Ra (A), positive ion MS-MS spectrum obtained for MNa⁺ molecular ion peak at m/z 1175 by using MALDI-TOF/TOF (B) and positive ion MS-MS spectrum obtained for MNa⁺ molecular ion peak at m/z 1203 using MALDI-TOF/TOF (C).

FIG. 8. Structure of α-MAME from M. tuberculosis H37Ra. It is a methyl ester of α-alkyl, β-hydroxy fatty acid and is present as a homologous series differing by 28 amu in H37Ra. The two MAMEs we investigated were those where a=17, b=10, c=17 and 19, and d=23. For c=17 the calculated molecular weight is 1155.2 (C₇₉H₁₅₈O₃). For c=19 the calculated molecular weight is 11183.3 (C₈₁H₁₆₂O₃). These molecular weight values were 1.0030 greater than the corresponding molecular weights given by MALDI mass spectrometry where PEG and PPG were used as standards. The major fragments in MS-MS are due to the loss of —OH group (−17) and —OCH₃ group (methyl ester, −31).

FIG. 9. Negative ion MALDI mass spectrum of B1(A) and negative ion MS-MS spectrum of the M-H⁻ peak at m/z 1234 obtained by MALDI-TOF/TOF (B).

FIG. 10. The proposed structure of the major component of B1 and B1-3 where M=1235.2 (C₈₂H₁₅₄O₆).

FIG. 11. MALDI mass spectra of (A) B2 and (B) B3.

FIG. 12. Positive ion MS-MS spectra of m/z 1258 (A), 1244 (B) and 1202 (C) which were obtained from MALDI of B1-3, B2 and B3, respectively.

FIG. 13. TLC of B1 and saponified B1. Lane-1, 20 μg B1; lane-2, 20 μg of saponified B1; lane-3, 10 μg tetracosanoic acid. Silica gel plate and petroleum ether/diethyl ether/acetic acid (7:1:0.1) solvent were used.

FIG. 14. TLC of B1 treated with acetyl-chloride. Lane-1, 20 μg B1; lane-2, 20 μg acetylated B1. Silica gel plate and CMN (92:8:0.8) solvent were used.

FIG. 15. TLC of B1 treated with diazomethane. Lane-1, 20 μg B1; lane-2, 20 μg methylated B1; lane-3, 10 μg MAME. Silica gel plate and PE/E (7:1) solvent were used.

FIG. 16. MALDI mass spectrum of B1-3.

### DETAILED DESCRIPTION

### Definitions

It is to be appreciated that certain aspects, modes, embodiments, variations and features of the invention are described below in various levels of detail in order to provide a substantial understanding of the present invention. The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a,” “an” and “the” include plural referents unless the content clearly dictates otherwise. For example, reference to “a lipid” includes a combination of two or more lipids, and the like.

As used herein, phrases such as element A is “associated with” element B mean both elements exist, but should not be interpreted as meaning one element necessarily is causally linked to the other.

As used herein, the “administration” of an agent, drug, or compound to a subject includes any route of introducing or delivering to a subject a compound to perform its intended function. Administration can be carried out by any suitable route, including orally, sublingually, intranasally, parenterally (intravenously, intramuscularly, intraperitoneally, or subcutaneously), or topically. In particular embodiments, administration is pulmonary. In particular embodiments, pulmonary administration comprises intratracheal instillation, intratracheal delivery of liposomes, insufflation, nebulization, dry powder inhalation, aerosol inhalation, or bronchoalveolar lavage. In some embodiments, pulmonary administration comprises the use of a propellant. In some embodiments, the propellant comprises hydrofluoroalkanes, chlorofluorocarbons, propane, nitrogen, or a mixture thereof.

As used herein, “Mycobacterium biofilm” refers to biofilms produced by Mycobacterium or comprising Mycobacterium. The term encompasses biofilms comprising Mycobacterium alone, and Mycobacterium in combination with other pathogenic or non-pathogenic microbes. In some embodiments, the biofilm comprises a single species of Mycobacterium. In some embodiments, the biofilm comprises multiple species of Mycobacterium. In some embodiments, the biofilm is associated with an active Mycobacterium infection. As used herein, the term encompasses in vivo and in vitro biofilms.

As used herein, the terms “effective amount,” “therapeutically effective amount,” and “pharmaceutically effective amount” refer to a quantity sufficient to achieve a desired therapeutic and/or prophylactic effect. For example, an effective amount is one which results in the prevention or reduction of Mycobacterium infection, or one or more symptoms associated therewith. In the context of therapeutic or prophylactic applications, the amount of a composition administered to the subject will depend on the type and severity of the disease and on the characteristics of the individual, such as general health, age, sex, body weight and tolerance to drugs. The skilled artisan will be able to determine appropriate dosages depending on these and other factors. Where the compositions described herein are administered in combination with one or more additional therapeutic compounds, the skilled artisan will take this into consideration when determining the effective amount.

As used herein, “prevention” or “preventing” of a disorder or condition refers to a therapy-induced reduction in the occurrence of the disorder or associated symptoms relative to an untreated control sample or subject. The term encompasses delayed onset, slowed progression, and/or reduced severity of the disorder or associated symptoms relative to an untreated control condition. For example, in some embodiments, a therapeutic composition is administered to a subject exposed to Mycobacterium, or at risk of Mycobacterium infection, either before exposure or before signs or symptoms of Mycobacterium infection have developed.

As used herein, the term “subject” and “patient” are used interchangeably, and refer to a member of any vertebrate species. The methods of the presently disclosed subject matter are particularly useful for warm-blooded vertebrates, including humans. The methods provided herein are also useful for the treatment of non-human mammals such those typically kept as pets, as well as those mammals of other importance due endangerment, economic importance (such as agricultural importance), or social importance (such as pets or captive animals).

As used herein, the terms “treating” or “treatment” or “alleviation” refers to therapeutic treatment, wherein the object is to prevent the onset of, slow the progression of, reduce the severity of, or ameliorate the symptoms of a targeted pathologic condition or disorder. For example, a subject is successfully “treated” for Mycobacterium infection, if after receiving a therapeutic amount of a therapeutic composition according to the methods described herein, the subject shows observable and/or measurable reduction in or absence of one or more signs or symptoms of the infection. As used herein, the terms encompass substantial reduction of signs or symptoms associated with the pathogenic condition or disorder (e.g., a Mycobacterium infection), which includes partial and total reduction, wherein some medically or biologically relevant result is achieved.

As used herein, “disrupting” a biofilm produced by Mycobacterium refers to aiding in the dispersal of and/or inhibiting the formation of the biofilm. The term encompasses the inhibition or prevention of de novo biofilm formation as well as the dispersal of an established biofilm. As used herein, the term encompasses all degrees of biofilm disruption, including complete disruption, substantially complete disruption, and partial disruption. The term encompasses a reduction in the rate of biofilm formation, the dimensions of the biofilm, and/or the amount of material incorporated into the biofilm. In some embodiments, biofilm disruption occurs in the lung of a subject infected with Mycobacterium. In some embodiments, biofilm disruption occurs in the course of treating Mycobacterium infection. In some embodiments, biofilm disruption occurs in the course of administering anti-microbial agents to a subject in need thereof.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

The present disclosure relates to the disruption of microbial biofilms. In particular, the present technology provides methods for the disruption of biofilms using compounds similar to those produced by Mycobacterium sp., such as compounds produced by M. smegmatis. In practicing the present technology, many conventional techniques of chemistry, biochemistry, and microbiology are used. These techniques are well-known in the art and are provided in any number of available publications, including the following: The Prokaryotes: Symbiotic Associations, Biotechnology, Applied Microbiology, Martin, Ed., Springer (2006); Bioremediation Protocols, Vol. 2, Sheehan, Ed., Humana Press (1997); Methods in Microbiology, Vol. 19, Bergan, Ed., Elsevier Science (1984); Norris and Ribbons, Methods in Microbiology, Academic Press (1971); Immunochemical Methods in Cell and Molecular Biology, Mayer and Walker eds. (1987) (Academic Press, London); Weir's Handbook of Experimental Immunology, Herzenberg, et al., eds (1996).

The present lipid compounds can be prepared by reacting a phthiocerol-related diol, e.g., a diol of the formula shown below (where “Prot” is an appropriate protecting group, such as a benzyl group), where “n” is an integer and R″ is a lower alkyl group, with activated acyl

CH₃—(CH₂)ₙ—CH(OR)—CH₂—CH(OR′)—(CH₂)₄—CH(CH₃)—CH(OProt)-R″

compound(s). Examples of suitable activated acyl compounds include acyl chlorides and N-hydroxysuccinimide esters. After esterification with the activated acyl compound(s), the protecting group can be selectively removed to provide a hydroxydiester compound. The activated acyl compounds are typically derived from an unsaturated fatty acid, e.g., polyunsaturated fatty acid and/or an omega-3 fatty acid, and/or a polymethyl substituted fatty acid, group, e.g., a mycoserosic acid. The diol may be reacted with a mixture of such activated acyl compounds or one of the hydroxy groups in the diol depicted above may be selectively protected to permit sequential esterification of the two internal hydroxy groups.

Examples of suitable compounds which can be produced using the above methods include compounds having the formula:

CH₃—(CH₂)ₙ—CH(OR)—CH₂—CH(OR′)—(CH₂)₄—CH(CH₃)—CH(OH)—R″

in which R″ is typically a lower alkyl group, such as a methyl or ethyl group; and one of R and R′ is an unsaturated fatty acyl group and the other is a polymethyl substituted fatty acyl group (such as an acyl group derived from a mycocerosic acid).

The compounds of the present technology may be prepared as natural products isolated from in vitro cultures of M. smegmatis. Methods for culturing the bacteria are known in the art, and include but are not limited to those described in the Examples below. The bacteria may be grown, for example, as a planktonic culture in 7H9 broth supplemented with albumin-dextrose-catalase (7H9/ADC), or glycerol-alanine-salts (GAS) media. Likewise the bacteria may be serially cultured in these media. The bacteria may be cultured for such a time as necessary to reach late log phase, with bacterial growth monitored using methods known in the art. The culture may then be harvested and prepared for storage or further processing using methods known in the art, such as washing the bacterial pellet with a neutral buffer, filtering the culture supernatant, and cold-temperature storage.

In some embodiments, M. smegmatis is initially cultured in 7H9 broth supplemented with 10% (v/v) albumin-dextrose-catalase (ADC) for 16 h at 37° C., and then cultured to late-log phase (absorbance at 650 nm of >1.5) in glycerol-alanine-salts (GAS) medium in the incubator/shaker at 37° C. and 150 rpm. In some embodiments, the cells are harvested by centrifugation, washed with cold phosphate-buffered saline (PBS), and the cell pellet (CP) from centrifugation were stored in the freezer at −80° C. until processed further. In some embodiments, supernatant fraction was filtered (CF), methanol was added to 1.0% and stored at 0-5° C.

Compounds of the present technology may be isolated from M. smegmatis cultures using aqueous and organic solvents as known in the art, including but not limited to acetone, chloroform, methanol, or mixtures thereof. In some embodiments, the compounds are extracted from bacterial pellets or culture supernatants using chloroform/methanol at a ratio of about 1:1, about 2:1, about 3:1, about 4:1, about 5:1, about 6:1, about 7:1, about 8:1, about 9:1, about 10:1. In some embodiments, the CM extract is dried and further extracted with acetone. In some embodiments, bacterial pellets or culture supernatants are extracted using a methanol/chloroform at a ratio of about 1:1, about 2:1, about 3:1, about 4:1, about 5:1, about 6:1, about 7:1, about 8:1, about 9:1, about 10:1. In some embodiments, bacterial pellets or culture supernatants are extracted using methanol/water at a ratio of about 1:1, about 2:1, about 3:1, about 4:1, about 5:1, about 6:1, about 7:1, about 8:1, about 9:1, about 10:1.

In some embodiments, compounds of the present technology are serially extracted from bacterial pellets using methanol, chloroform/methanol (CM), and chloroform/methanol/ammonium hydroxide (CMN). In some embodiments, CM comprises a ratio of 2:1. In some embodiments, CMN comprises ratios of 99:1:0.1, 98:2:0.2, 96:4:0.4, 94:6:0.6, or 90:10:1.

Compounds of the present technology extracted from bacterial pellets or culture supernatants may be isolated and/or purified using methods known in the art, including but not limited to chromatography. In some embodiments, isolation and/or purification comprise thin layer chromatography (TLC). The skilled artisan will understand that methods for TLC are known in the art and generally comprise the separation of compounds on a solid medium using one or more solvents or solvent systems. In some embodiments, the solid medium comprises silica gel. In some embodiments, the solvent system comprises chloroform/methanol/concentrated ammonium hydroxide (CMN), petroleum ether/diethyl ether/acetic acid, petroleum ether/diethyl ether, or hexane/diethyl ether/acetic acid.

In some embodiments, products isolated by TLC are further isolated and/or purified by solid-phase extraction. The skilled artisan will understand that solid-phase extraction comprises application of one or more compounds to a solid medium and selective elution of the compound from the medium using one or more solvents or solvent systems. In some embodiments, the solid medium comprises a silica gel column. In some embodiments, the solid medium comprises C₁₈-bonded silica gel cartridges. In some embodiments, the solvent or solvent system comprises hexane/diethyl ether/acetic acid, methanol, methanol/water, or methanol/chloroform. In some embodiments, the cartridge is pre-conditioned with methanol and serially eluted with methanol/water (2:1), methanol, methanol/chloroform (4:1), methanol/chloroform (1:1).

Compounds of the present technology isolated and/or purified from M. smegmatis may be evaluated for the capacity to inhibit biofilm formation using methods known in the art, including but not limited to those provided in the Examples below. For example, biofilm-forming bacteria may be cultured in vitro under conditions permissive for biofilm formation, and a formulation of compounds of the present technology added to the culture. The effect of the compounds on the bacterial biofilm may be assessed by comparison to control samples not exposed to the compound. The compounds may be added to the culture prior to the formation of a biofilm in order to assess the capacity of the compound to prevent the formation of a biofilm. Alternatively, the compound may be added to the culture after the formation in order to assess the capacity of the compound to disrupt the biofilm or inhibit growth or expansion of the biofilm.

The formula and/or chemical structure of the compounds of the present technology may be assessed using methods known in the art, including but not limited to MALDI mass spectrometry and tandem mass spectrometry (MS-MS). Methods for obtaining and interpreting such data are readily available in the art, including those provided in the Examples below.

### Methods of Treatment

Disclosed herein are pharmaceutical compositions useful for preventing or disrupting a Mycobacterium biofilm, such as in the prevention or treatment of Mycobacterium infection in a subject in need thereof. Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., multiple days of treatment).

Pharmaceutical compositions suitable for injectable use can include sterile aqueous solutions (where the components are water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J., USA), or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or serotes; a glidant such as colloidal silicon dioxide; a sweetening agent such as sucrose or saccharin; or a flavoring agent such as peppermint, methyl salicylate, or orange flavoring.

The pharmaceutical compounds described herein may be formulated for pulmonary administration, such as, for example, by intratracheal instillation, intratracheal delivery of liposomes, insufflation, nebulization, dry powder inhalation, aerosol inhalation, bronchoalveolar lavage, or other methods such as known in the art. Depending on the form of pulmonary administration, the compound may be formulated, as droplets, liquids, powders, liposomes, or other forms such as known in the art. In some instances, the formulation may comprise one or more propellants, such as hydrofluoroalkanes, chlorofluorocarbons, propane, nitrogen, or other propellants as known in the art. Formulations for pulmonary administration may be optionally be formulated for controlled release, such as known in the art.

Dosage, toxicity and therapeutic efficacy of compositions described herein can be determined by standard pharmaceutical procedures in cell cultures or experimental animals. Such methods may be used to determine lethal dose to 50% of a population (LD₅₀) and the dose therapeutically effective in 50% of a population (ED₅₀). The therapeutic index is typically expressed as the LD₅₀/ED₅₀ ratio. Compositions which exhibit high therapeutic indices are generally preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a target tissue concentration range that includes the IC₅₀ (i.e., the concentration of the test compound which achieves a half-maximal inhibition of symptoms) as determined in cell culture. Such information can be used to more accurately determine useful doses in humans. Compound concentrations in target tissues or plasma can be measured, for example, high performance liquid chromatography.

In some embodiments, an effective amount of a composition described herein is one sufficient for achieving a therapeutic or prophylactic effect. In some embodiments, the dose ranges from about 0.000001 mg per kilogram body weight per administration to about 10,000 mg per kilogram body weight per administration. Suitably, the dosage ranges are from about 0.0001 mg per kilogram body weight per administration to about 100 mg per kilogram body weight per administration. Administration can be provided as an initial dose, followed by one or more additional doses. Additional doses can be provided at any time deemed appropriate following the initial dose. For example, the period of time between the initial dose and one or more additional doses may be on the order of seconds, minutes, hours, days, weeks, months, or years. In some embodiments, one or more additional doses are administered after an evaluation of the subject's target tissue or plasma concentration of the composition. In some embodiments, one or more additional doses are administered after the extent or severity of the subject's illness is re-evaluated using methods known in the art.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

Pharmaceutical compositions described herein may be administered alone or in conjunction with one or more anti-bacterial compounds known in the art. In some embodiments, the anti-bacterial compound is an anti-mycobacterial compound. In some embodiments, the anti-mycobacterial compound comprises isoniazid, rifampicin, pyrazinamide, ethambutol, or a combination thereof. The skilled artisan will understand that selection of an anti-mycobacterial compound to be administered in conjunction with compounds of the present technology will depend on various factors including but not limited to, the severity of the disease or disorder, previous treatments, the general health and/or age of the subject, and other diseases present.

The therapeutic and/or pharmaceutical compositions described here may be administered to a subject before or after exposure to a pathogen such as Mycobacterium. For example, in some embodiments, a therapeutic composition is administered to a subject exposed to Mycobacterium, before signs or symptoms of Mycobacterium infection have developed. In some embodiments, a therapeutic composition is administered to a subject or at risk of Mycobacterium infection before exposure to the pathogen, during exposure and/or after exposure to the pathogen to prevent infection or at least to alleviate the symptoms of infection. In some embodiments, a therapeutic composition is administered to a subject suffering from Mycobacterium infection. In some embodiments, additional doses are provided to a subject to prevent recurrence of symptoms or reduce severity of a recurrence of symptoms.

### EXAMPLES

The following examples are provided by way of illustration to further describe the subject matter disclosed herein and are not intended to limit the scope of the claims. All percentages are by weight unless otherwise noted.

### Example 1

### Planktonic M. Smegmatis Produces a Novel Structural Series of Hexadecenoyl-/Mycocerosyl-Phthiotriol Diesters that Inhibit its Own Biofilm Formation

### Methods

Reagents.

All solvents used in this study were reagent grade. Acetyl-chloride was purchased from Aldrich, Milwaukee Wis., USA. Hexacosanoic acid and 1-hexadecanol were purchased from Analabs, North Haven, Conn., USA. Diazomethane was generated from N-methyl-N-nitroso-p-toluenesulfonamide (Diazald, Sigma, St. Louis, Mo., USA). Mycolic acids were prepared from the cell wall skeleton of M. smegmatis as previously described for Corynebacterium matruchotii. Mycolic acid methyl ester (MAME) was prepared by treating purified mycolic acids dissolved in chloroform/methanol (C/M, 2:1) with fresh diazomethane. Trehalose monomycolate (TMM) was isolated and purified from M. smegmatis and characterized by matrix-assisted laser desorption/ionization (MALDI) mass spectrometry.

Bacterial Strain and Growth Condition.

M. smegmatis mc²155 obtained from Del Besra (Birmingham, UK) was initially cultured in 7H9 broth supplemented with 10% (v/v) albumin-dextrose-catalase (ADC, Becton Dickinson, Sparks, Md., USA) for 16 h at 37° C. It was then cultured to late-log phase (absorbance at 650 nm of >1.5) in glycerol-alanine-salts (GAS) medium in the incubator/shaker at 37° C. and 150 rpm. These are planktonic (submerged growth) cells. The cells were harvested by centrifugation, washed with cold phosphate-buffered saline (PBS) and the cell pellet (CP) from centrifugation were stored in the freezer at −80° C. until used. The supernatant fraction was filtered (CF), methanol was added to 1.0% and stored at 0-5° C.

Silica gel Thin-Layer Chromatography (TLC).

TLC was performed on pre-scored silica gel GHL (250 μm) (Analtech, Newark, Del., USA). For the analysis of the biologically active lipids, the solvent systems were chloroform/methanol/concentrated ammonium hydroxide (CMN, 85:15:1.5), (90:10:1) and (92:8:0.8). The solvent system for TLC of free fatty acids was petroleum ether/diethyl ether/acetic acid (7:1:0.1). The solvent system for TLC of fatty acid methyl ester (FAME) and MAME was petroleum ether/diethyl ether (PE/E, 7:1). The lipid bands on the plate were detected by spraying with 0.6% dichromate in 55% sulfuric acid followed by charring.

Isolation and Partial Purification of the Lipid in M. Smegmatis which Disperses Biofilm of M. smegmatis.

M. smegmatis was cultured in GAS medium (4 L) in the incubator/shaker at 37° C. to an absorbance at 650 nm of about 1.5 and harvested by centrifugation to yield a CF and a CP. The CF was acidified with HCl, extracted twice with 300 ml of chloroform and the pooled extract was dried. The resulting residue was extracted with acetone to yield an acetone-soluble fraction (744 mg). This fraction was partially purified on a silica gel column (Merck, grade 62) using hexane/diethyl ether/acetic acid (7:1:0.1) solvent system to yield a minor fraction associated with fatty acids (4.9 mg, designated CF-lipid) that was tested for dispersing mature M. smegmatis biofilm.

The CP of M. smegmatis was extracted three times with 50 ml of CM (2:1) and the pooled extract was dried. This residue was extracted with acetone to yield an acetone-soluble fraction (273 mg) which was partially purified on a silica gel column as described above for CF-lipid to yield a pooled fraction (61.3 mg, designated CP-lipid) that was also tested for dispersing mature M. smegmatis biofilm.

Isolation and Purification of the Biologically Active Lipid from CF by Solid Phase Extraction.

The M. smegmatis CF (966 ml) was loaded onto ten-1 gram C₁₈-bonded silica gel cartridges (Varian Mega Bond Elut) preconditioned in methanol and the cartridges were eluted in order with 10 ml each of (a) methanol/water (2:1); (b) methanol; (c) methanol/chloroform (M/C, 4:1); and finally (d) M/C (1:1). Each of these fractions was collected separately, pooled, dried and weighed: methanol/water (2:1) effluent, 462.0 mg; methanol effluent, 17.7 mg; M/C (4:1) effluent, 11.0 mg; and M/C (1:1) effluent, 12.9 mg. Each of these four fractions was analyzed by TLC and tested for M. smegmatis biofilm inhibition.

Purification of the M/C Effluents by Preparative TLC.

The M/C (4:1) effluent and M/C (1:1) effluent were each loaded onto several 10×10-cm silica gel GHL plates at 1 mg/cm along with 50 μg of the corresponding samples on the left and right side strips. The plates were developed in the CMN (90:10:1) solvent. With the central part of the plate protected with a glass plate covered with aluminum foil, the side strips were sprayed with dichromate/sulfuric acid reagent and lightly charred with a heat gun. The expected positions of the bands at the center of the plate were thus determined. The silica gel strips at the central part of the plate which corresponded to the bands on the side strips were marked with a pencil, scraped from the plate, extracted with CM, 2:1 and dried. The M/C (4:1) effluent yielded 1.4 mg of a single band and the M/C (1:1) effluent yielded 3.1 mg of a broad band. Both of the bands were near the solvent front. These fractions were analyzed by TLC and tested for inhibition of M. smegmatis biofilm formation.

Isolation and Purification of the Biologically Active Lipid from the CP.

Frozen CP of M. smegmatis (14.22 g wet weight) from 16 L of cell culture were thawed and extracted with 300 ml of CM (2:1) with stirring at room temperature for 24 h. This mixture was filtered and the residue was extracted twice in the same manner. The pooled extract was evaporated to complete dryness on a rotary evaporator and under high vacuum. The dried CM extract was further extracted twice with 50 ml of methanol to yield (a) CM-soluble/methanol-insoluble (sample A, 550 mg) and (b) CM-soluble/methanol-soluble (sample B, 460 mg) fractions. Sample A (highly polar) was set aside and sample B was dried and extracted twice with 50 ml of CM (2:1) to yield 230 mg of a red wine-colored material. This sample was applied to a 2×17-cm silica gel column in chloroform and the column was washed with 100 ml of CMN (99:1:0.1). The following shallow step-wise gradient was then used for the fractionation: (a) 150 ml of CMN (98:2:0.2); (b) 200 ml of CMN (96:4:0.4); and finally (c) 100 ml of CMN (94:6:0.6). The collection of 5 ml fractions began as the colored band came off the column. Aliquots (20 μl) of a total of 65 fractions were analyzed by TLC using the solvent system of CMN (90:10:1). Based on the TLC results, the following fractions were pooled, dried and weighed: #25-29, designated B, 43 mg; #30-38, B2, 16.4 mg; #39-61, B3, 14.3 mg; and #62-65, B4, 6.3 mg.

Purification of B1 by Preparative TLC.

B1 (8.0 mg) was further fractionated on a 10×10-cm silica gel GHL plate at 1.3 mg/cm along with 50 μg of B1 on the left and right side strips and developed in the CMN (90:10:1) solvent. The position of the bands on the plate was determined as previously described for preparative TLC, recovered and dried. Going from origin to front, the dry weights for the bands were as follows: B1-1, 2.6 mg; B1-2, 2.4 mg; and B1-3, 1.9 mg.

Assay for M. smegmatis Biofilm Inhibition.

Two ml of 7H9/ADC culture medium was inoculated with 20 μA of a starter culture of M. smegmatis mc²155 in 40% glycerol and incubated at 37° C. without shaking for 16-24 hr. This culture was transferred into 10 ml of GAS medium and incubated in the incubator/shaker at 37° C. to an absorbance at 650 nm of <1.5, diluted to an absorbance of 0.02-0.03 in M63 culture medium. Then aliquots of 2.0 ml of this inoculated M63 medium were placed in sterile 1.5×10-cm glass tubes and the purified compounds to be tested were added. The test tubes were incubated in the heat block at 37° C. for 3 to 7 days. Pellicles began to form at the air-liquid interface of control culture on day-2, the pellicle thickened and cells began climbing up the surface of the glass. Such pellicles are recognized as biofilm. In the biofilm inhibition assay, the purified compounds dissolved in CM (2:1) were added at zero time and the development of biofilm was recorded on day-3 and -4.

MALDI Mass Spectrometry and Tandem Mass Spectrometry (MS-MS).

MALDI experiments were performed on a Bruker Ultraflex III mass spectrometer (Billerica, Mass., USA) equipped with a SMART BEAM laser, a LIFT cell and Compass v.1.2 software. The sample dissolved in CM (2:1 or 1:1) were placed on top of a thin layer of 2,5-dihydroxybenzoic acid on the stainless steel target. Calibration spots were prepared by placing PEG 1500 or PPG 1000 in methanol on top of the thin layer of 2,5-dihydroxybenzoic acid close to the sample location. Fragmentation (MS-MS) was generated by raising the laser power and the potential of the LIFT cell.

### Results

Compounds Produced by M. smegmatis Can Disperse Mature M. smegmatis Biofilm.

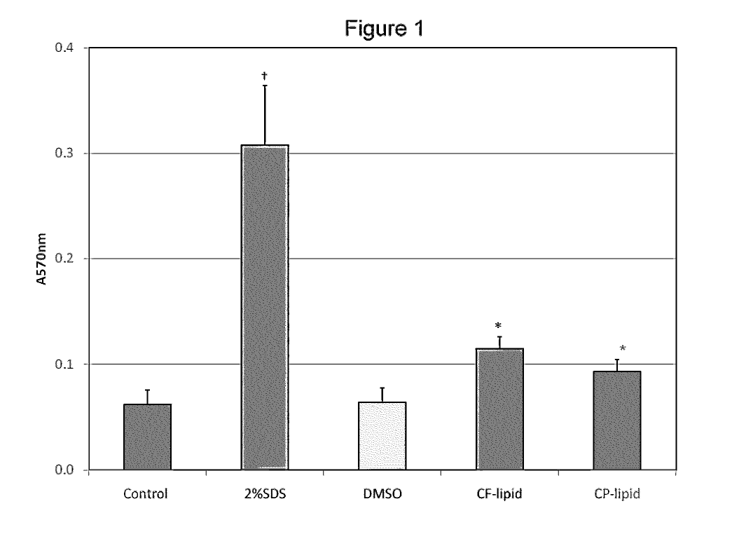

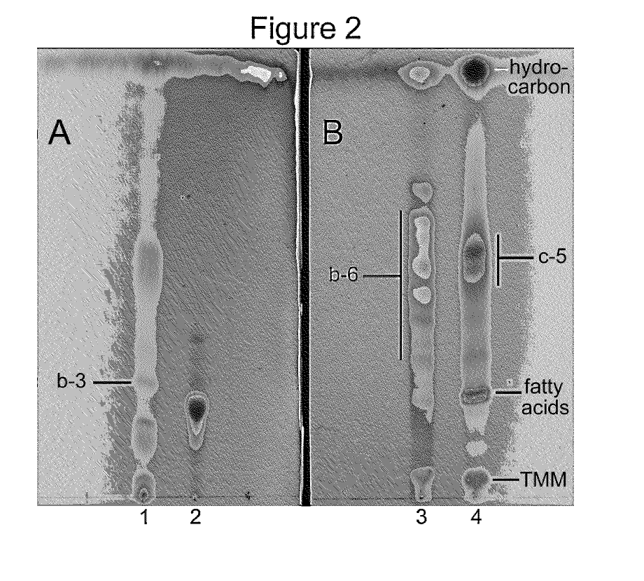

Partially purified lipids from the CF and CP of M. smegmatis designated CF-lipid and CP-lipid were dissolved in dimethyl sulfoxide (DMSO) and tested in the biofilm dispersion assay used by Davies and Marques (Davies, D. G., and C. N. Marques. 2009. A fatty acid messenger is responsible for inducing dispersion in microbial biofilms. J. Bacteriol. 191:1393-1403.) The results are shown in FIG. 1. At the 200 μg/ml concentration, both CF-lipid and CP-lipid were active in dispersing mature M. smegmatis biofilm with P>0.05 for CF-lipid and CP-lipid versus the DMSO control. This is first evidence for the presence of the an active compound in M. smegmatis that affects biofilm formation.

Isolation and Purification of Inhibitors of Biofilm Formation in the CF of M. smegmatis.

The chemical signal to stop biofilm formation in Mycobacteria should accumulate in the culture medium as the cells grow and mature. This signal would stop biofilm formation allowing planktonic cells to begin colonizing at new sites. We thus investigated the CF of a mature planktonic culture of M. smegmatis for the presence of such a compound(s). The CF (966 ml) was processed using solid phase extraction to obtain (a) methanol/water (2:1), (b) methanol, (c) M/C (4:1) and (d) M/C (1:1) effluent fractions. Each fraction was pooled and dried. The calculated yields of the methanol, M/C (4:1) and M/C (1:1) effluents were 18, 11 and 13 μg/ml of CF, respectively. These are minor components of the CF. TLC analysis of these latter three fractions showed that they are complex mixtures (FIG. 2).

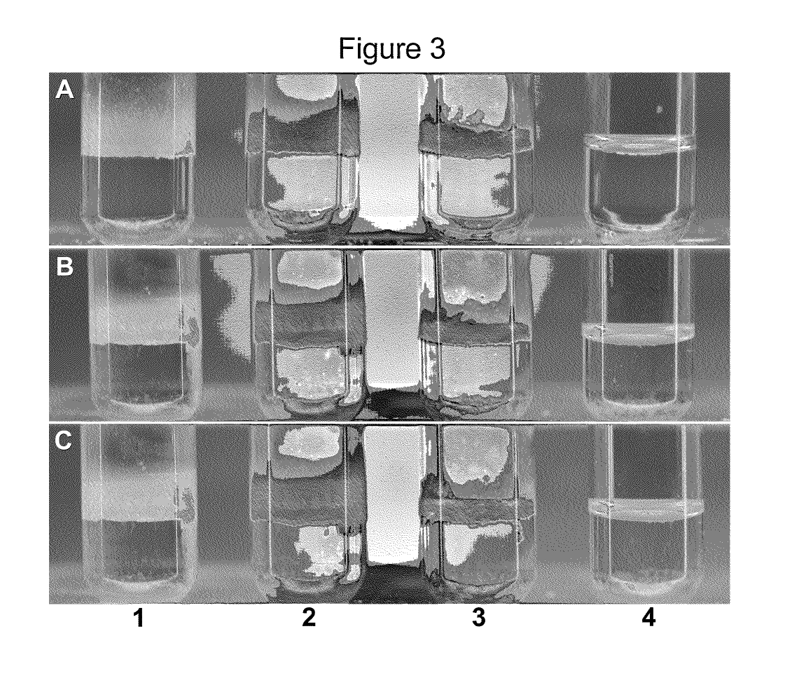

In a preliminary small-scale experiment, we found that only the methanol, M/C (4:1) and M/C (1:1) effluents were active in inhibiting M. smegmatis biofilm formation at 100 μg/ml (data not shown). So we purified these three cartridge fractions from a large-scale preparation by preparative TLC. We obtained 1.4 mg of a fast-moving TLC-7 band from 11.0 mg of M/C (4:1) effluent. We also obtained fast moving TLC-6 (1.7 mg) and -7 (1.4 mg) bands from 12.9 mg of M/C (1:1) effluent fractions. The M. smegmatis biofilm inhibition assay showed that M/C (4:1)/TLC-7 fraction was active at 30 μg/ml and 60 μg/ml (FIG. 3A). M/C (1:1)/TLC-6, -7 were both active at 30 μg/ml and 60 μg/ml (FIG. 3B-3C). We confirmed that a higher concentration of methanol effluent is required to show full biological activity. We were able to associate the biological activity to a TLC band that we termed b-3 in the methanol effluent, to b-6 with multiple bands in the M/C (4:1) effluent and to c-5 with a double band in the M/C (1:1) effluent (FIG. 2). The M/C (1:1) effluent also contained a hydrocarbon at the solvent front, fatty acids as a double band and TMM but these were not active.

Isolation and Purification of Inhibitors of Biofilm Formation from the CP of M. smegmatis.

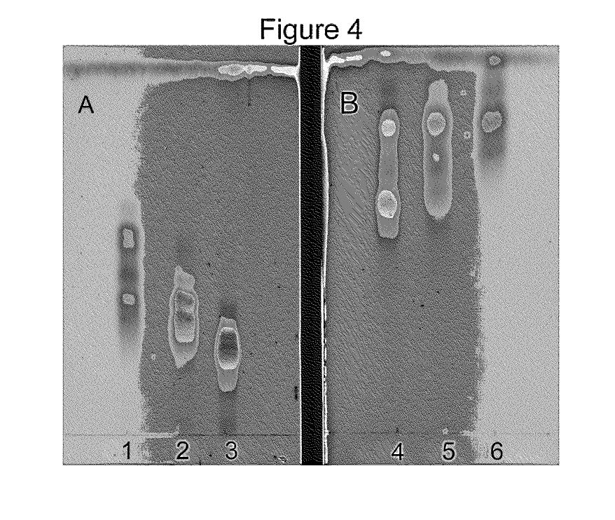

Since we found compounds that inhibit M. smegmatis biofilm formation in the CF to be quite minor, we speculated that the CP would be a better source. The CP of M. smegmatis (14.22 g wet weight) was extracted with CM (2:1) and the extract was dried. The dried extract was then re-extracted with methanol and dried (sample B). This sample B was then extracted with CM (2:1) to yield a CM-soluble/methanol-soluble/CM-soluble fraction (230 mg). TLC analysis of this sample showed that it is related to M/C (4:1) and M/C (1:1) effluent fractions from the CF. Thus, this sample was fractionated on a silica gel column and we obtained pooled fractions B1, B2, B3 and B4. The highly polar B4 fraction was set aside since it was not active. Analytical TLC of B1, B2 and B3 are shown in FIG. 4A. As shown in FIG. 4A, these fractions are complex mixtures containing multiple bands. On TLC, they co-migrated with biologically active b-6 and c-5 which are found in the CF. The biologically active b-3 from CF was associated with the CM-soluble/methanol-insoluble/CM-soluble fraction of CM-extract.

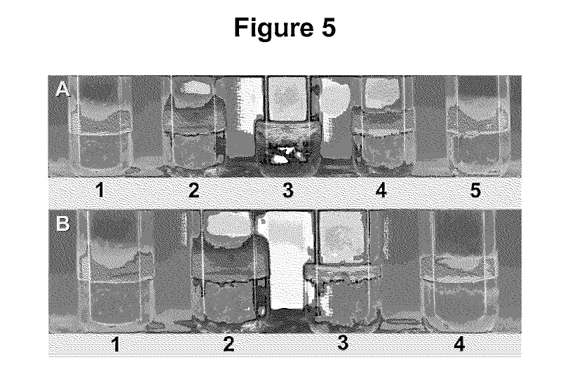

The results of M. smegmatis biofilm inhibition assay of B1 is shown in FIG. 5A. B1 was fully active at 25 μg/ml and partially active at 12.5 μg/ml. B1 and B2 were similarly active (data not shown).

The major silica gel column fraction B1 was further purified by preparative TLC to yield sub-fractions B 1-1, B1-2 and B1-3 (from origin to the solvent front). Analytical TLC of these fractions showed that B 1-3 is enriched in the fast-moving band and B 1-1 is enriched in the slow-moving band of B1 (FIG. 4B). B1-3 was devoid of the prominent slow-moving band found in B1-1. Both B1-1 and B1-3 were shown to be active in inhibiting M. smegmatis biofilm formation at 12.5 μg/ml (FIG. 5B). The B1-3 fraction corresponded to biologically active c-5 lipid in the M/C (1:1) effluent of the CF (FIG. 2). B1-1 was active, but B1-2 was not tested. We are assuming that it is also biologically active since it contains the major fast-moving band found in B1-3 and the prominent slow-moving band in B 1-1. B1-2 appears to be enriched in minor band appearing in the middle of the chromatogram which might also be active.

In the biofilm inhibition assay, control culture began developing a smooth surface growth after two days and the cells began to climb up the wall of the tube. The surface growth continued and thickened over days-4 and -5. There was no observable submerged growth in the M63 medium over this period. When purified c-5 was added to the assay tube at day-0 of incubation, no surface growth was observed up to 3 day. After this period, rough surface pellicles began to appear along with some submerged growth (planktonic growth). The addition of B1, B2 and B3 on day-0.5 and day-1 of incubation delayed the onset of biofilm formation.

Chemical Analyses of B1 and B2.

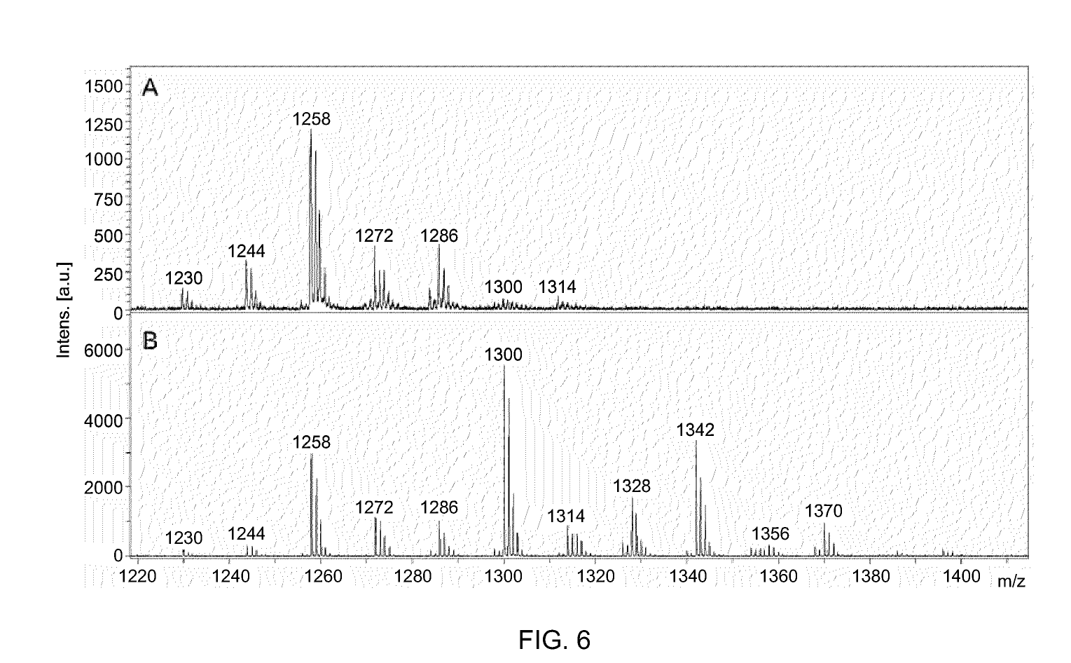

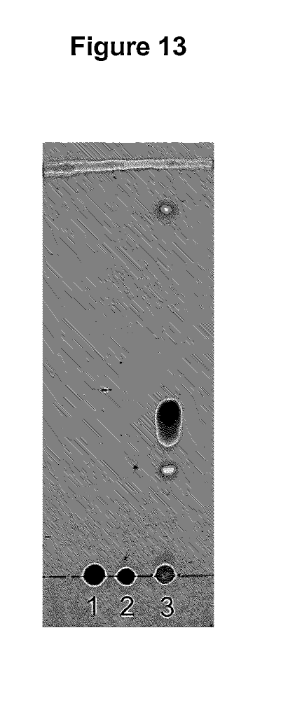

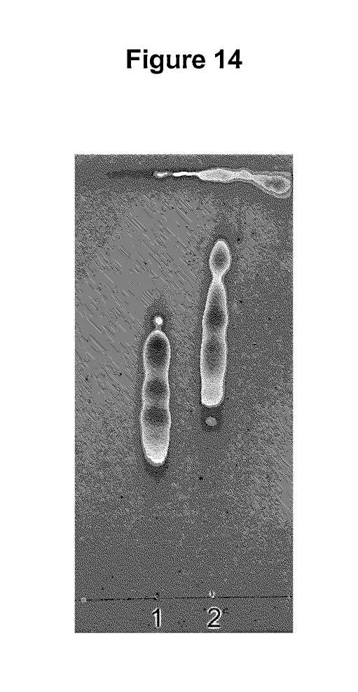

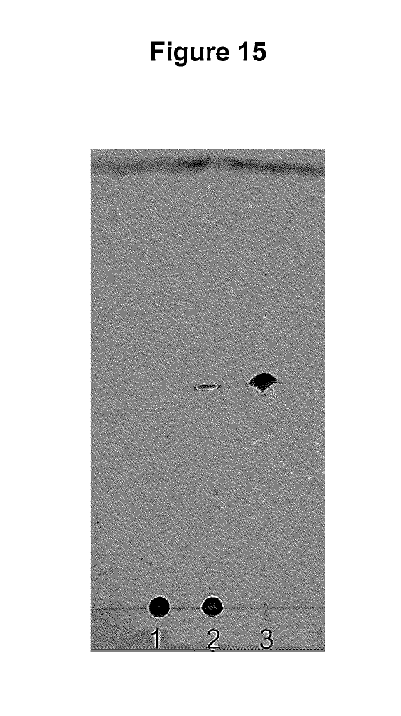

Samples B1 and B2 were analyzed by several chemical methods. Since B3 was still a crude sample based on analytical TLC and MALDI analysis, the results of the chemical analyses were inconclusive. Both B1 and B2 did not methylate with diazomethane showing that the compounds in these two samples do not contain the free carboxyl group and that they are not free mycolic acids. This was illustrated with B1 (FIG. 13). B1 was readily derivatized with acetyl-chloride to the acetate ester based on both analytical TLC (FIG. 14) and MALDI mass spectrometry, suggesting that the compounds in B1 contain at least one free hydroxyl group. FIG. 14 shows that the mobility of the TLC bands in B1 increases after acetylation. FIG. 6 shows two positive ion MALDI spectra where a major molecular ion MNa⁺ appears at m/z 1258 before acetylation and this increases to m/z 1300 after acetylation. This increase is 42 amu or the size of one acetyl group [CH₃—C(O)-1]. The appearance of a prominent peak at m/z 1342 in the spectrum of the acetylated B1 suggests that a minor component with two free hydroxyl group(s) exist(s). Finally, analytical TLC of saponified B1 showed that a fatty acid is liberated from the compound (FIG. 15). We did not identify the fatty alcohol product of this hydrolysis reaction (the presumed alkyl polyol) by TLC. However products of partial hydrolysis were visible and recognizable by TLC.

Fragmentation Pattern of α-MAME from M. tuberculosis H37Ra by Positive Ion MS-MS.

Tandem mass spectrometry used in this study is a relatively new and powerful technology that can separate μg quantities of a complex mixture of compounds and analyze each component separately. Tandem mass spectrometry of α-MAME using MALDI has not been previously reported.

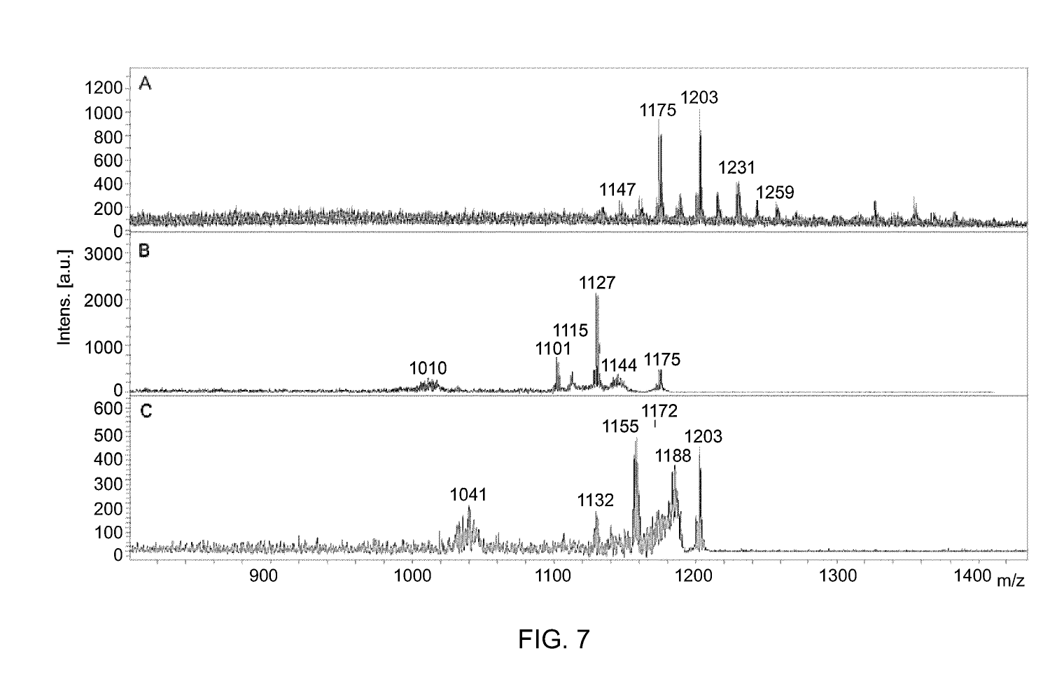

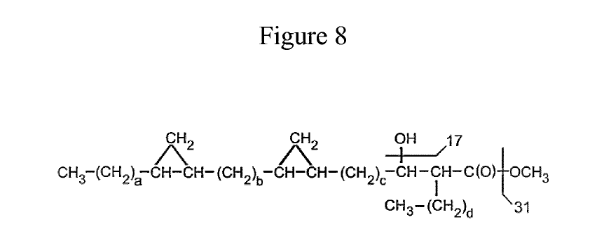

HPLC-purified α-MAMEs from M. tuberculosis H37Ra were tested as a model compound by positive ion MALDI mass spectrometry and MS-MS. The results are shown in FIG. 7. As expected, the positive ion MALDI spectrum (FIG. 7A) showed a homologous structural series of quasi-molecular ions MNa⁺ at m/z 1147, 1175. 1203, 1231 and 1259 that differed by 28 amu. The structures of this series of MAMEs are established (FIG. 8). Positive ion MS-MS spectrum of the m/z 1175 peak was obtained using a MALDI-TOF/TOF (FIG. 7B). The spectrum showed fragment ions at m/z 1144 (MNa⁺−31), 1127 (MNa⁺−31−17), 1115 (MNa⁺−31−29), 1101 (MNa⁺−31−43) and a broad peak centered at 1011 (MNa⁺−17−141/155, MNa⁺−17−31−113 and MNa⁺−169).

Positive ion MS-MS spectrum of the larger m/z 1203 peak was obtained using a MALDI-TOF/TOF (FIG. 7C). The spectrum showed fragment ions at m/z 1186 (MNa⁺−17), 1172 (MNa⁺−31), 1155 (MNa⁺−17−31) and 1132 (MNa⁺-71), and a broad peak centered at 1041 (MNa⁺−17−155 and MNa⁺−31−113).

These two MS-MS spectra showed that the α-MAMEs fragments yield MNa⁺ ion minus —OCH₃ (31 amu) and —OH (17 amu) groups. Additionally, there we numerous fragmentations at different and unspecified points on either or both alkyl side chain at the distal end (15 to 169 amu). Fragmentation did not occur around the cyclopropane ring. There was no indication that pyrolysis had occurred (the cleavage of the α-alkyl, β-hydroxy linkage to generate a fatty aldehyde and FAME) which is common in electron impact mass spectrometry of MAME. These results were verified by analyzing the highly purified α-, α′-MAMEs from M. smegmatis which contains unsaturation rather than cyclopropane ring (data not shown).

Negative ion MALDI Mass Spectrum of B1 and Negative ion MS-MS Spectrum of the Major Molecular Ion.

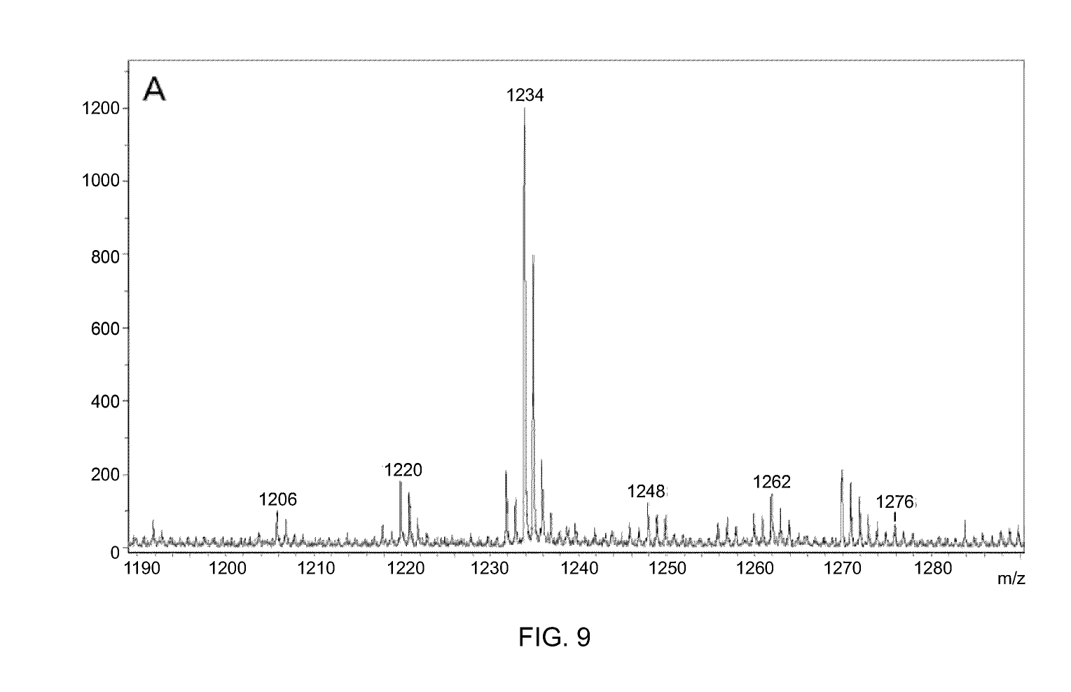

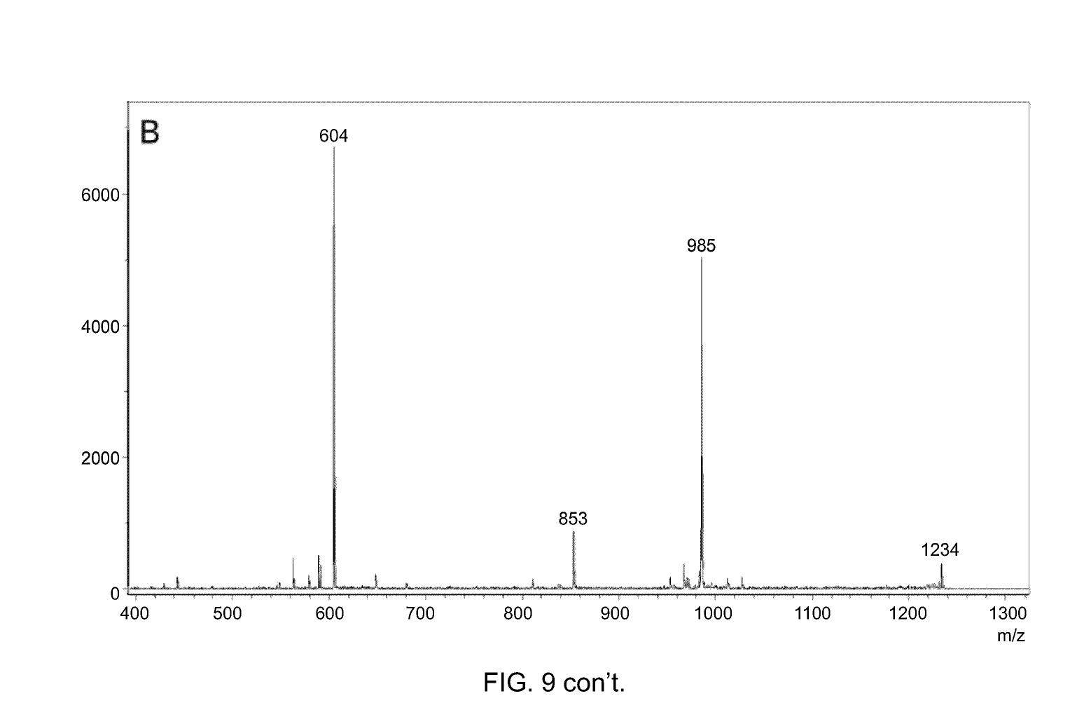

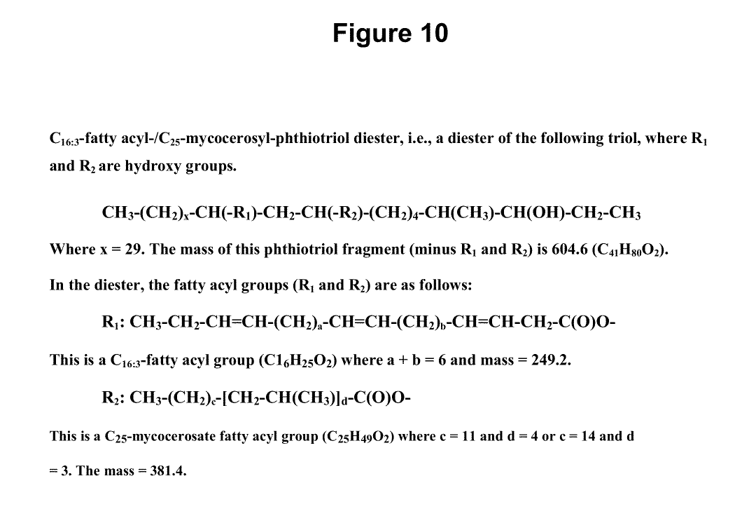

The B1 sample was analyzed by negative ion MALDI and the results are shown in FIG. 9A. The spectrum showed M-H⁻ molecular ion peaks at m/z 1206, 1220, 1234, 1248, 1262 and 1276 at 14 amu intervals. Negative ion MS-MS spectrum of the m/z 1234 peak was obtained using a MALDI-TOF/TOF (FIG. 9B). It showed that the M-H⁻ molecular ion had fragmented into three clearly defined pieces. No other prominent cleavage was observed. The spectrum showed the original molecular ion at m/z 1234 and fragment ions at m/z 985, 853 and 604. These were cleavages of the ester linkages. We have identified these fragments as follows: M−1−249 for 985, M−1−381 for 853 and M−1−249-381 for 604. The 249 fragment would be the C_{16:3}-fatty acyl group, 381 fragments would be the C₂₅-mycocerosyl group and the 604 fragment would then be the alkyl polyol that carries the two fatty acids in ester linkages. The proposed structure of this novel compound (the M-H⁻ peak at m/z 1234) is given in FIG. 10. It is a C_{16:3}-fatty acyl-/C₂₅-mycocerosyl-phthiotriol diester.

MALDI Mass Spectrum of B1-3.

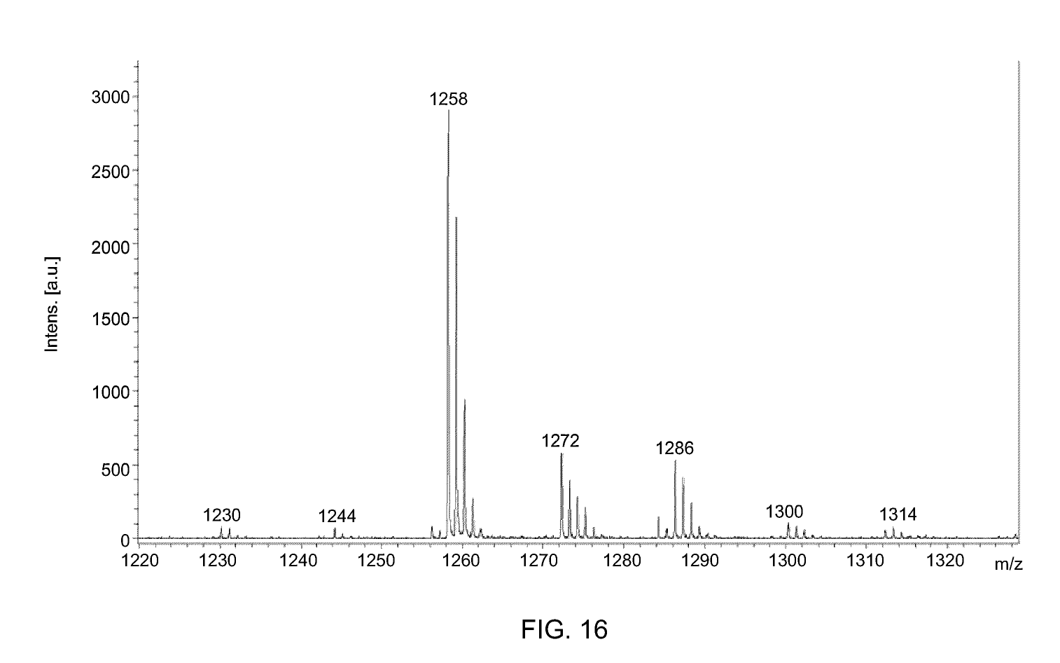

The silica gel column purified B1 from the CM (1:1) extract of whole cells of M. smegmatis was further purified by preparative TLC to yield a B1-3 fraction which was the major component of B1 based on TLC (FIG. 4B). This fraction was analyzed by MALDI mass spectrometry. The spectrum showed molecular ion peaks MNa⁺ at m/z 1230, 1244, 1258, 1271, 1286, 1300 and 1314 (FIG. 16). When compared to the MALDI spectrum of B1, the peak at m/z 1258 was more prominent relative to the other peaks in the spectrum suggesting that preparative TLC had enriched this m/z 1258 component.

MALDI Mass Spectra of Silica Gel Column Fractions B2 and B3.

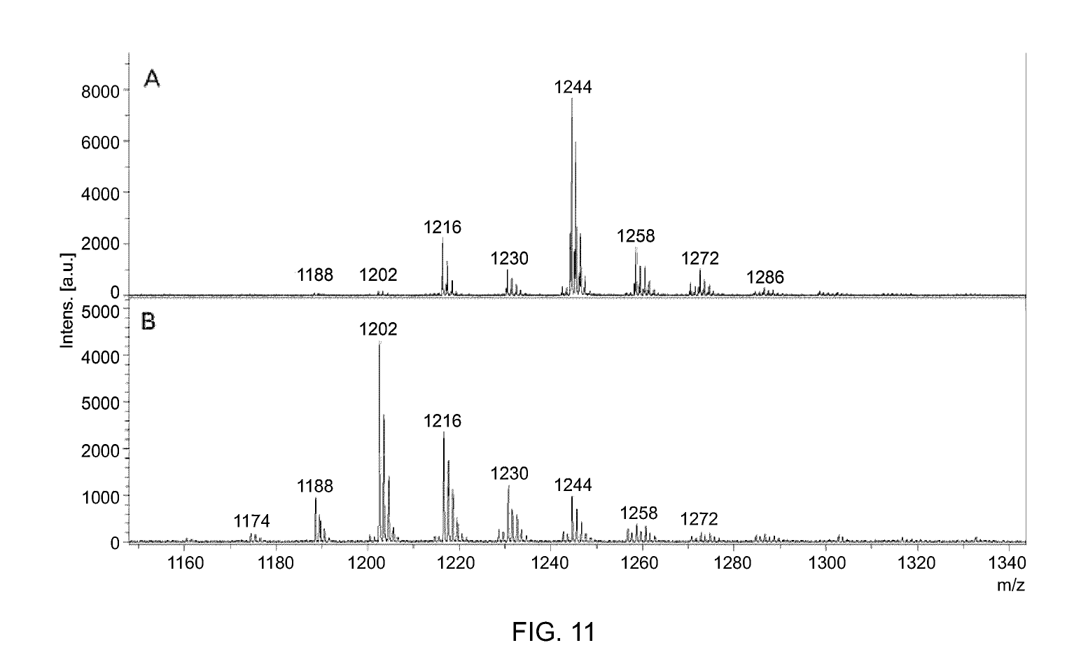

MALDI spectrum of B2 (FIG. 11A) showed the same structural series as in B1 except that they were smaller in size (MNa⁺ at m/z 1188, 1202, 1216, 1230, 1244, 1258, 1272, 1286 and 1300) with maximum intensity at m/z 1245. FIG. 11B shows that the same structural series in B3 was even smaller in size (MNa⁺ at m/z 1174, 1188, 1202, 1216, 1230, 1244, 1258 and 1272) with maximum intensity at m/z 1202. B2 and B3 appear to extend down the structural series from B1. Thus MALDI shows that B1-3, B2 and B3 contain MNa⁺ peaks at 14 amu intervals representing a structural series of phthiotriol diesters with a wide range of sizes from m/z 1174 to 1314.

Positive ion MS-MS Analysis of the Major MALDI Peak in B1-3, B2 and B3.

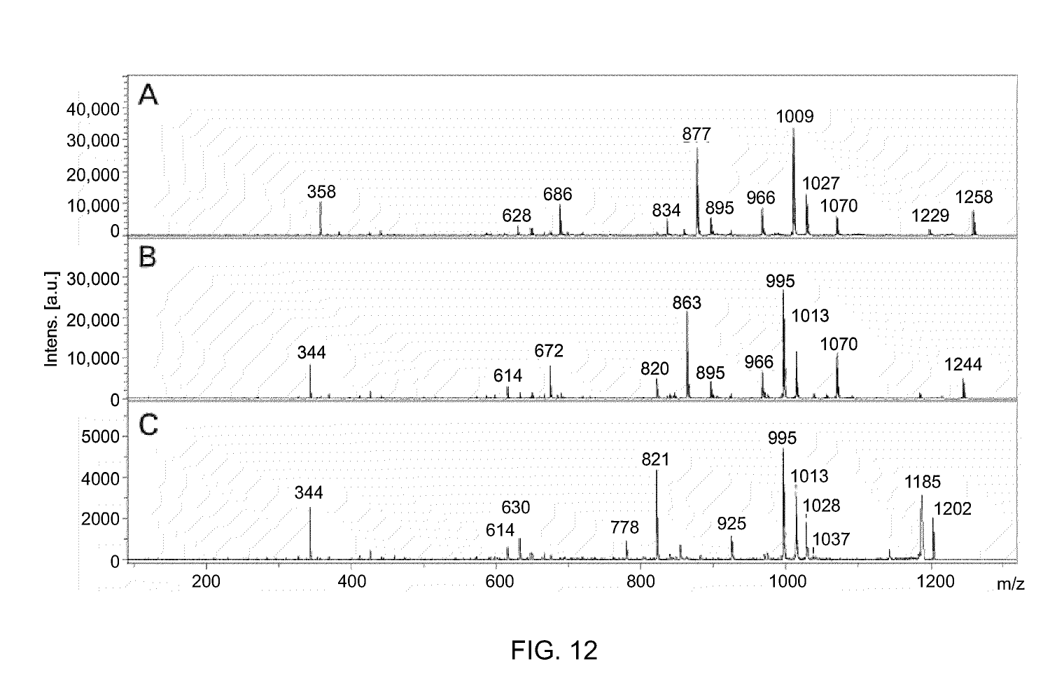

Positive ion MS-MS spectra of the MALDI peaks at m/z 1258 from B1-3, 1244 from B2 and 1202 from B3 were obtained using the MALDI-TOF/TOF (FIG. 12). Each of the fragments formed from the MS-MS of the m/z 1258 component were first identified (Table 1). The most significant fragments were at m/z 1009 (1258−249; a loss of C_{16:3}-fatty acyl group), 877 (1258−381: a loss of C₂₅-mycocerosyl group) and 628 (1258−249−381; loss of both C_{16:3}-fatty acyl and C₂₅-mycocerosyl groups). Other important fragments included m/z 1070 (1258−129−59), 1027 (1258−249+18) and 686 (1258−249−381+58). Fragments at m/z 686 and 1229 allowed us to tentatively assign the position of the distal and proximal unsaturations in the C_{16:3}-fatty acyl group.

Similar fragments were identified from the positive ion MS-MS spectra of m/z 1244 and 1202 and their sizes were compared with those from the m/z 1258 component (Table 2). In summary, it showed that the 1258 component differed from the 1244 component by 14 amu which was a difference in the value of x in the proposed structure (FIG. 10). Thus for the 1258 component, x=29 and for the 1244 component, x=28. Compared to the 1258 component, the 1202 component was 14 amu smaller in the 604 fragment and 42 amu smaller (isopropyl unit) in the 381 fragment. Thus it has x=28, c=11 and d=3 or x=28, c=10 and d=2 in the structure. It also confirms that the m/z 381 fragment is the mycocerosyl acyl group. Unspecified cleavages of the long-chain hydrocarbons also occurred at the distal positions.

Assuming that the molecular weight of the major active component of B1 and B1-3 (C_{16:3}-fatty acyl-/C₂₅-mycocerosyl-phthiotriol diesters) is 1235 (FIG. 10), complete inhibition of M. smegmatis biofilm formation at 12.5 μg/ml would be 0.8 μM.

TABLE 1
Identification of the fragments in positive ion MS-MS
spectrum of the MNa⁺ molecular ion at m/z 1258 peak
obtained using MALDI-TOF/TOF. M = 1235. Refer to
FIG. 12A. The vertical arrow indicates the point of cleavage.
The major fragment ion is given in bold face lettering.

| Peak | Assignment | Fragment released |
|---|---|---|
| 1258 | M + 23 | None |
| 1229 | 1258 − 29 | CH₃—CH₂—↑CH═CH— |
| | | of C_{16:3}-fatty acyl |
| 1070 | 1258 − 129 − 59 | HO—(CH₂)₈—↑CH₂— - and - |
| | | —CH₂—↑CH(OH)—CH₂—CH₃ |
| 1027 | 1258 − 249 + 18 | C_{16:3}-fatty acyl (and hydration) |
| 1009 | 1258 − 249 | C_{16:3}-fatty acyl |
| 966 | 1258 −249 − 43 | C_{16:3}-fatty acyl and |
| | | CH₃—CH₂—CH₂—↑CH₂— - |
| 895 | 1258 − 381 + 18 | C₂₅-mycocerosyl (and hydration) |
| 877 | 1258 − 381 | C₂₅-mycocerosyl |
| 834 | 1258 − 381 − 43 | C₂₅-mycocerosyl and |
| | | CH₃—CH₂—CH₂—↑CH₂— - |
| 686 | 1258 − 249 − | - —O—C(O)—CH₂—↑CH═CH—CH₂— - |
| | 381 + 58 | of C_{16:3}-fatty acyl |
| 628 | 1258 − 249 − 381 | C_{16:3}-fatty acyl and C₂₅-mycocerosyl |
| 358 | 627 − 269 | HO—(CH₂)₁₈—↑CH₂— - |
| | | of 604 + 23 fragment |

### Discussion

In our initial attempt to find a biologically active compound(s) in M. smegmatis, we examined the CF and CP as the possible sources. We extracted the CF with chloroform and CP with CM (2:1) and partially purified them by silica gel column chromatography to obtain CF-lipid and CP-lipid, respectively. Both fractions were found to be active at 200 μg/ml using the biofilm dispersion assay. The procedure of chloroform extraction of CF was cumbersome and the biofilm dispersion assay involves too many steps for its preparation to be practical. Thus, we decided to use solid-phase extraction of CF and the much simpler and basic biofilm inhibition assay in our study.

Using the C₁₈-bonded silica gel cartridges for the CF of M. smegmatis, we recovered three fractions that were biologically active using the biofilm inhibition assay: (a) methanol effluent, (b) M/C (4:1) effluent, and (c) M/C (1:1) effluent. We identified the active components we designated as b-3 in the methanol effluent, b-6 in the M/C (4:1) effluent and c-5 in the M/C (1:1) effluent. The b-3 was isolated, purified and fully characterized but it will not be discussed here. The unidentified b-6 shows a complex TLC pattern but it could be part of c-5. The M/C (4:1) effluent and M/C (1:1) effluent were both further fractionated by preparative-TLC to yield M/C (4:1)/TLC-7, M/C (1:1)/TLC-6 and -7 fractions which were all biologically active. The M/C (1:1)/TLC-7 fraction is our highly purified c-5 fraction from CF.

We proceeded to investigate the CP as a better source of the biologically active compound in M. smegmatis. The CM-extract of the CP was dried and extracted with methanol and fractionated on a silica gel column to yield fractions B1, B2, and B3. All three fractions were active in the M. smegmatis biofilm inhibition assay. B1 was further purified by preparative-TLC to yield B1-1, B1-2 and B1-3 of which B1-3 was the major component (equivalent to c-5 from the CF based on TLC). When B1, B2, and B3 were acetylated with acetyl-chloride, their mobility on TLC increased, suggesting that they all contained free hydroxyl group. When B1, B2, and B3 were saponified and analyzed by TLC, it showed that they decomposed to fatty acids and fatty alcohol. The fatty acid was not mycolic acid. These purified fractions from the CP of M. smegmatis were used for structural determination by both negative ion and positive ion MALDI and MS-MS.

Silica gel column fractions B2 and B3 were also analyzed by MALDI mass spectrometry and the spectra revealed that they are an extension of the same structural series of B1 to the lower masses. The three fractions (B1, B2 and B3) contained overlapping molecular ion peaks and they extended down from m/z 1314 for B1 to m/z 1174 for B3. Thus they are composed of the same structural series of lipids with a wide range of sizes differing by 14 amu.

The B1 fraction was analyzed by negative ion MALDI mass spectrometry and the spectrum revealed a major molecular ion at M-H— at m/z 1234. This molecular ion was then analyzed by negative ion MS-MS and the spectrum clearly showed three prominent fragments at m/z 985, 853 and 604. From these fragments, we deduced that this molecule contains C_{16:3}-fatty acyl (1234−985=249), and C₂₅-mycocerosyl-(1234−853=381) groups and an alkyl polyol (1234−249−381=604). Compound A of phthiotriol closely fits the description of this alkyl polyol but the latter is longer (x=29 versus 20, 22). The C_{16:3}-fatty acid is an unusual component of this molecule.

The phthiotriol along with phthiocerol and phthiodolone are found in either the A or B form in M. tuberculosis and M. bovis. The phthiocerol occur as diesters of mycocerosic acids called DIM. The mycocerosic acid in M. tuberculosis is 2, 4, 6, 8-tetramethyloctacosanoic acid. We might speculate that the phthiotriol and phthiodolone diesters of mycocerosic acids are also present along with DIM but as minor components. Nothing is known regarding the location or function of DIM or phthiotriol in the cell. The presence of diesters of phthiotriol in M. smegmatis is a novel observation.

TABLE 2
Comparing the size of the identified peaks in the positive ion
MS-MS spectra of MNa⁺ at m/z 1258, 1244 and 1202 (three
chosen peaks of the structural series). The nature of each fragment
for MNa⁺ at m/z 1258 from positive ion MS-MS analysis listed in
the first column was determined as shown in Table 1. The corresponding
fragments for MNa⁺ at m/z 1244 and 1202 from the MS-MS
analysis are listed in the second and third columns, respectively
(FIG. 12B, 12C). The last column lists the size differences
relative to the fragments for 1258.

| | | | —CH₂ group in 1245 |
|---|---|---|---|
| Peak | −Δ14 | −Δ56 | and 1203 |
| 1258 | 1244 | 1202 | −Δ14 and −Δ56 |
| 1070 | 1070 | 1028 | same and −Δ42 |
| 1027 | 1013 | 1013 | −Δ14 and −Δ14 |
| 1009 | 995 | 995 | −Δ14 and −Δ14 |
| 877 | 863 | 821 | −Δ14 and −Δ56 |
| 686 | 672 | 630 | −Δ14 and −Δ56 |
| 628 | 614 | 614 | −Δ14 and −Δ14 |
| 358 | 344 | 344 | −Δ14 and −Δ14 |

### Example 2

### Disruption of M. Tuberculosis Biofilm Production by a Novel Structural Series of Hexadecenoyl-/Mycocerosyl-Phthiotriol Diesters Produced by M. Smegmatis

The example will demonstrate that a novel structural series of hexadecenoyl-/mycocerosyl-phthiotriol diesters produced by M. smegmatis disrupts biofilms produced by M. tuberculosis.

Two ml of 7H9/ADC culture medium will be inoculated with 20 μl of a starter culture of M. tuberculosis in 40% glycerol and incubated at 37° C. without shaking for 16-24 hr. This culture will be transferred into 10 ml of GAS medium and incubated in the incubator/shaker at 37° C. to an absorbance at 650 nm of <1.5, and diluted to an absorbance of 0.02-0.03 in M63 culture medium. Then aliquots of 2.0 ml of this inoculated M63 medium will be placed in sterile 1.5×10-cm glass tubes and the purified compounds to be tested will be added. The test tubes will be incubated in the heat block at 37° C. for 3 to 7 days. Pellicles are expected to form at the air-liquid interface of control culture on day-2. The pellicles are expected to thicken and climb the surface of the glass as illustrated in FIGS. 3 and 5. Such pellicles are recognized as biofilm.

The purified compounds will be dissolved in CM (2:1) and added to the biofilm cultures at zero time. The level of biofilm development will be recorded on days 3 and 4 following addition of the purified compounds. It is expected that the addition of the purified compound to the biofilm cultures will slow the continued development of the biofilms and/or cause a reduction in the size of the pre-existing biofilm.

### Example 3

### Methods of Treating M. Tuberculosis Infection in Guinea Pigs

This example will demonstrate that the pharmaceutical compositions described herein are useful in the prevention or treatment of Mycobacterium infection in animal subjects.

### Materials and Methods

Guinea Pigs.

Female outbred Hartley guinea pigs (weight, 500 g) are purchased from the Charles River Laboratories (North Wilmington, Mass.) and are held under barrier conditions in a biosafety level 3 animal laboratory. The specific-pathogen-free nature of the guinea pig colonies is demonstrated by testing sentinel animals.

Experimental Infections in Guinea Pigs.

Guinea pigs are infected (n=5) using a Madison chamber aerosol generation device, which delivers approximately 20 M. tuberculosis strain Erdman K01 bacilli into the lungs. The animals are harvested and analyzed for bacterial load determinations (n=5), histopathology (n=5), and flow cytometric analysis (n=4) on days 25, 50, 75, 100, 125, and 150 days of the infection. As described previously, the bacterial loads in the organs of the guinea pigs at each time point of the study are determined by plating serial dilutions of homogenates of lungs (right cranial lobe), spleen, and mediastinal lymph node tissues on nutrient Middlebrook 7H11 agar and counting the numbers of CFU after 6 weeks of incubation at 37° C. The bacterial load for each organ is calculated and converted to logarithmic units. The data will be expressed as the mean log₁₀ number of CFU±the standard error of the mean for each group.

Treatment Protocol.

On day 20 of the infection, four guinea pigs are euthanized to determine the bacterial load prior to the start of drug treatment on day 25. The remaining animals are randomly assigned to two groups: 25 guinea pigs in the control group and 60 guinea pigs in the drug treatment group. Pharmaceutical compositions are then administered 5 days a week for the duration of the 19-week treatment regimen in the form of an aerosol formulated with a suitable propellant. Animals in the control group are treated with the propellant only. Animals in the experimental group are treated with an active compound such as described herein. Doses are chosen based on previous pharmacokinetic and pharmacodynamic studies performed with this species. The animals are carefully monitored on a daily basis using the Karnovsky scale as known in the art. Assisted feeding is administered as necessary as the infection progresses.

At the conclusion of the 19-week treatment period, randomly selected subjects are taken off the drug therapy. These animals are then euthanized; and their lungs, spleens, and mediastinal lymph nodes (MLNs) removed. The number of infectious organisms present is determined by plating serial dilutions of 10-ml organ tissue homogenates on nutrient Middlebrook 7H11 agar plates (Gibco BRL, Gaithersburg, Md., USA). Bacterial colonies are counted after 3 to 4 weeks of incubation at 37° C.

Histological analysis. The left caudal lung lobe, spleen, and lymph nodes from each guinea pig (n=5) is collected at necropsy and fixed in 4% paraformaldehyde in phosphate-buffered saline (PBS). Randomly selected tissue sections are embedded in paraffin and cut to 5 lam on a microtome. The tissue sections are mounted on glass slides, deparaffinized and stained with hematoxylin-eosin and carbolfuchsin by the Ziehl-Neelsen method, as reported previously. The lung and lymph node lesion areas relative to the normal tissue area are quantified by a stereology-based method, referred to as the area fraction fractionator, with the investigator blind to all treatment groups. The stereology workstation consists of a Nikon 80i research microscope equipped for bright-field and fluorescence microscopy with chromatic aberration-free infinity (CFI) objectives (×2/0.01 Plan Apo, ×4/0.2 Plan Apo, ×10/0.30 Plan Fluor, ×20/0.75 Plan Apo, ×40/0.75 Plan Fluor, ×100/1.40 Plan Apo), a three-axis computer-controlled stepping stage with linear grid encoders, a z-axis motorized specimen stage for automated sampling, a personal computer with a frame grabber board, a color digital camera, a 24-in. monitor, and stereology and virtual slice zoomify software (StereoInvestigator, version 8.2; MBF Bioscience, Williston, Vt.) with the following: three-dimensional (3D) serial section reconstruction, solid modeling, virtual slice, and confocal and magnetic resonance imaging (MRI) modules. The lung and lesion areas on representative hematoxylin-eosin-stained sections are determined. In addition, acid-fast-stained sections are evaluated. The area of inflammation relative to the area of normal tissue parenchyma is estimated from representative lung, lymph node, and spleen tissue sections evaluated at ×20 magnification. A total of 8 to 12 fields are randomly selected by the computer, and a counting frame (2,000 μm2) containing probe points with a grid spacing of 200 μm is used to define the areas of interest (lesions and lungs). The data is expressed as the mean ratio of the lesion area to the lung area for all the animals within a treatment group. A lesion shown in a photomicrograph represents the section that is closest to the mean value for each group.

MRI.

Four lungs are harvested from the animals before the start of treatment, and thereafter, the lungs of two guinea pigs in each group are harvested at the indicated time points. At the time of euthanasia, the lungs are inflated with 20 ml of room air per kg of body weight by tracheal intubation. The pulmonary vasculature is flushed free of blood with 60 ml of phosphate buffered saline and perfusion fixed with 60 ml of 4% paraformaldehyde via the right ventricle. Once the lungs are perfusion fixed, they are removed en bloc and immersion fixed for 1 week in 4% paraformaldehyde. After immersion fixation, the heart and esophagus of each animal are dissected from the lungs. The lungs and attached mediastinal lymph nodes are embedded in 4% low-melting-point agarose in phosphate-buffered saline. The embedded lungs are then scanned by MRI. The lungs are harvested from the control guinea pigs on days 29, 50, 73, and 100 following exposure to M. tuberculosis and from the drug-treated guinea pigs on days 29, 50, 78, 105, and 134 following exposure to M. tuberculosis. MRI scans are performed with a 1.5-T Signa 9.1 LX magnetic resonance instrument (General Electric Medical Systems, Milwaukee, Wis., USA), with the specimens being placed in a phased-array extremity coil. A T1-weighted 3D volume scan is acquired (repetition time, 20 ms; echo time, 6.4 ms; slice thickness, 1.2 mm; field of view, 6 cm) for an in-plane spatial resolution of 234 μm. Images of the entire lung specimen obtained by MRI are evaluated by segmentation thresholding analysis with the Amira 2.3 program (Visage Imaging, Andover, Mass., USA). A line probe technique is used to select the boundaries between the signal intensities for healthy and diseased lungs. From that signal intensity boundary, a threshold value is used for automated selection of all tissues exhibiting that signal intensity and higher. Tissues such as heart and lymph nodes are segmented manually, and whole-lung tracings are also made to quantify the total sample size.

Flow Cytometry.

To prepare single-cell suspensions, the lungs and lymph nodes are perfused with 20.0 ml of a solution containing PBS and heparin (50 U/ml; Sigma-Aldrich, St. Louis, Mo., USA) through the pulmonary artery; and the caudal lobe and portions of the lymph nodes are aseptically removed from the pulmonary cavity, weighed, placed in medium, and dissected. In addition, leukocytes are separated from 10 ml of guinea pig blood as described before. The dissected lung tissue is incubated with complete Dulbecco modified Eagle medium (cDMEM) containing collagenase XI (0.7 mg/ml; Sigma-Aldrich) and type IV bovine pancreatic DNase (30 μg/ml; Sigma-Aldrich) for 30 min at 37° C. The digested lungs are further disrupted by gently pushing the tissue twice through a cell strainer (BD Biosciences, Lincoln Park, N.J.). Red blood cells are lysed with ammonium chloride-potassium (ACK) lysis buffer, washed, and resuspended in cDMEM. Total cell numbers are determined by flow cytometry with liquid counting beads, as described by the manufacturer (BD PharMingen, San Jose, Calif., USA).

Flow cytometric analysis of cell surface markers. Suspensions of single cells from the lungs and portions of the whole spleens and lymph nodes are prepared as described recently. Thereafter, suspensions of cells from each guinea pig are first incubated with CD4, CD8, pan-T-cell, CD45, MIL4, B-cell, macrophage, and class II antibodies at 4° C. for 30 min in the dark and after the cells are washed with PBS containing 0.1% sodium azide (Sigma-Aldrich). In addition, membrane permeabilization with Leucoperm permeabilizer (Serotec Inc, Raleigh, N.C.) is completed, according to the instructions of the manufacturer, before staining with macrophages and major histocompatibility complex (MHC) class II antibodies. Data acquisition and analysis is done with a FACscalibur fluorescence-activated cell sorter (FACS; BD Biosciences, Mountain View, Calif., USA) and CellQuest software (BD Biosciences, San Jose, Calif., USA). Compensation for the spectral overlap for each fluorochrome is done by using CD4, MIL4, or CD3 antigens from cells gated in the low forward scatter versus low side scatter, moderate to high forward scatter versus moderate to high side scatter, low side scatter versus MIL4-positive regions, high side scatter versus MIL4-negative regions, and high side scatter versus MIL4-positive regions. Analyses are performed with an acquisition of at least T cells 100,000 total events.

### Results

It is predicted that treatment with the pharmaceutical composition described herein will reduce the load of Mycobacterium in guinea pigs infected with the bacteria and reduce the incidence of lung lesions as measured by MRI. It is further predicted that such treatment will reduce the dissemination of pulmonary TB in infected subjects, as will be evident by histological evaluation of various organs. It is further predicted that such treatment will reduce the influx of T lymphocytes into the lungs, lymph nodes, and peripheral blood of infected subjects, as measured by flow cytometric detection CD4- and CD8-positive cells. It is similarly expected that such treatment will reduce the influx of MHC class II-positive macrophages into the lungs, lymph nodes, and blood. These results will demonstrate that the pharmaceutical compositions described herein are useful in the prevention and treatment of Mycobacterium infection in mammalian subjects.

### Example 4

### Methods of Treating M. Tuberculosis Infection in Humans

This example will demonstrate that the pharmaceutical compositions described herein are useful in the prevention or treatment of Mycobacterium infection in human subjects.

### Methods

Subjects:

A diagnosis of M. tuberculosis infection in human subjects is made using methods known in the art, including but not limited to sputum smear, chest x-ray, and tuberculin test. Subjects are randomized into control and experimental groups for 24 and 36 week treatment regimens. Baseline measurements of the extent and severity of Mycobacterium infection are recorded for each subject at prior to the start of the trial.

Treatment Protocol:

Patients take self-administered therapeutic compositions daily for 24 or 26 weeks, comprising isoniazid, rifampicin, pyrazinamide, ethambutol, or a combination thereof, alone or in conjunction with a pharmaceutical composition described herein. Pharmaceutical compositions are formulated for pulmonary administration, such as an aerosol or dry powder for inhalation, comprising a pharmaceutically acceptable propellant. At the conclusion of the treatment period, the extent and severity of each subject's Mycobacterium infection is re-evaluated by methods known in the art and compared to baseline measurements.

### Results

It is predicted that treatment with a pharmaceutical composition described herein will reduce the extent or severity of M. tuberculosis infection in human subjects, as evidenced by a decrease in the symptoms associated with Mycobacterium infection, and through measurement of disease indicators as known in the art. These results will demonstrate that the pharmaceutical compositions described herein are useful in methods of treating or preventing Mycobacterium infection in human subjects.

### Illustrative Embodiments

Reference is made in the following to a number of illustrative embodiments of the subject matter described herein. The following embodiments describe illustrative embodiments that may include the various features, characteristics, and advantages of the subject matter as presently described. These illustrative embodiments should not be considered as being comprehensive of all of the possible embodiments or as limiting the scope of the invention described herein.

In some embodiments, the present compound may suitably have a formula represented by:

CH₃—(CH₂)ₙ—CH(OR)—CH₂—CH(OR′)—(CH₂)₄—CH(CH₃)—CH(OH)—R″

wherein R″ is a lower alkyl group; n is an integer from 10 to 32 (commonly an integer from 24 to 32); one of R and R′ is a unsaturated fatty acyl group; and one of R and R′ is a polymethyl substituted fatty acyl group. Quite commonly, the lower alkyl group may be a methyl or ethyl group and, often, an ethyl group. The unsaturated fatty acyl group may be a polyunsaturated fatty acyl, which typically may have from 16 to 22 carbon atoms. For example, the unsaturated fatty acyl group may be a 16:3 fatty acyl group. The unsaturated fatty acyl group may be an omega-3 polyunsaturated fatty acyl group. The polymethyl substituted fatty acyl group may be an acyl group derived from a mycocerosic acid, e.g., a fatty acyl group represented by the formula:

CH₃—(CH₂)ₘ—(CH₂—CH(CH₃))ₓ—C(O)—

wherein m is an integer from 8 to 22; and x is 3 or 4. In certain embodiments, x may be 3 and m may be an integer from 12 to 17; and/or x may be 4 and m may be an integer from 9 to 14.

In some embodiments, the polyunsaturated fatty acyl group is represented by the formula:

CH₃—CH₂—CH═CH—(CH₂)ₐ—CH═CH—(CH₂)_{b}—CH═CH—CH₂—C(O)—

where a and b are integers and a+b=6.

In some embodiments, the omega-3 fatty acyl group is represented by the formula:

CH₃—CH₂—CH═CH—(CH₂)ₐ—CH═CH—(CH₂)_{b}—CH═CH—CH₂—C(O)—

where a and b are integers and a+b=6.

In some embodiments, the present lipid compounds may suitably have a formula:

CH₃—(CH₂)ₙ—CH(OR)—CH₂—CH(OR′)—(CH₂)₄—CH(CH₃)—CY(Z)—R″

in which R″ is typically a methyl or ethyl group; one of R and R′ is a polyunsaturated fatty acyl group; and one of R and R′ is an aliphatic acyl group having a formula:

CH₃—(CH₂)ₘ—(CH₂—CH(CH₃))ₓ—C(O)—

wherein m is an integer from 8 to 22; and x is 3 or 4. The polyunsaturated fatty acyl group may commonly have 16 or 18 carbon atoms. Examples of suitable polyunsaturated fatty acyl groups include 16:3 fatty acyl groups and 18:3 fatty acyl groups. The polyunsaturated fatty acyl group may be an omega-3 polyunsaturated fatty acyl group, such as a 16 carbon omega-3 fatty acyl group or an 18 carbon omega-3 fatty acyl group. These may include 16:3 omega-3 fatty acyl groups and/or 18:3 omega-3 fatty acyl groups. Specific examples of suitable polyunsaturated fatty acyl groups include a linoleoyl group and an alpha-linolenoyl group. The polyunsaturated fatty acyl group may be an all-cis polyunsaturated fatty acyl group, such as an all-cis 16:3 fatty acyl group. In some embodiments, the polyunsaturated fatty acyl group is represented by the formula:

CH₃—CH₂—CH═CH—(CH₂)ₐ—CH═CH—(CH₂)_{b}—CH═CH—CH₂—C(O)—

where a and b are integers and a+b=6.

In other embodiments, the present lipid compounds may suitably have a formula:

CH₃—(CH₂)ₙ—CH(OR)—CH₂—CH(OR′)—(CH₂)₄—CH(CH₃)—CH(OH)—R″

in which R″ is typically a methyl or ethyl group; one of R and R′ is an omega-3 fatty acyl group; and one of R and R′ is an aliphatic acyl group having a formula:

CH₃—(CH₂)ₘ—(CH₂—CH(CH₃))ₓ—C(O)—

wherein m is an integer from 8 to 22; and x is 3 or 4. The omega-3 fatty acyl group may be a 16 carbon omega-3 fatty acyl group or an 18 carbon omega-3 fatty acyl group (e.g., an omega-3 fatty acyl group derived from linolenic acid or linolenic acid). In some embodiments, the omega-3 fatty acyl group may be a polyunsaturated fatty acyl group, e.g., a 16:3 omega-3 fatty acyl group, an 18:2 omega-3 fatty acyl group and/or an 18:3 omega-3 fatty acyl group. In some instances, the omega-3 fatty acyl group may include an all-cis omega-3 polyunsaturated fatty acyl group. In some embodiments, the omega-3 fatty acyl group is represented by the formula:

CH₃—CH₂—CH═CH—(CH₂)ₐ—CH═CH—(CH₂)_{b}—CH═CH—CH₂—C(O)—

where a and b are integers and a+b=6.

In the present lipid compounds illustrated above, the aglycone portion of the compound may be based on a phthiocerol-related diester, e.g., the aglycone portion of a compound having the formula shown immediately below (where R and R′ are acyl groups). In such aglycone portions,

CH₃—(CH₂)ₙ—CH(OR)—CH₂—CH(OR′)—(CH₂)₄—CH(CH₃)—CH(OH)—R″

R″ is a lower alkyl group (i.e., a C1-C6 alkyl group), such as methyl or ethyl and “n” is an integer, commonly from 10 to 32. Typically, R″ may be an ethyl group. Quite often, “n” may be an integer from about 24 to 32.

In the present lipid compounds, the aliphatic acyl group may be a polymethyl substituted fatty acyl group related to a mycoserosic acid. The aliphatic acyl group may have about 22 to 32 carbon atoms, and often about 25 to 30 carbon atoms. For example, the aliphatic acyl group may be represented by the formula:

CH₃—(CH₂)ₘ—(CH₂—CH(CH₃))ₓ—C(O)—

wherein m is typically an integer from about 8 to 22 (often about 9 to 17); and x may be 3 or 4. In some embodiments, e.g., where “x” is 4, “m” may be an integer from about 9 to 14. In other embodiments, e.g., where “x” is 3, “m” may be an integer from about 12 to 17.

1. A compound having a formula:
CH₃—(CH₂)ₙ—CH(OR)—CH₂—CH(OR′)—(CH₂)₄—CH(CH₃)—CY(Z)—R″
wherein R″ is a lower alkyl group;
n is an integer from 10 to 32;
Y is —H; Z is —OH or —OCH₃; or Y and Z together form a carbonyl group with the carbon atom to which they are attached;
one of R and R′ is an unsaturated fatty acyl group; and
one of R and R′ is a polymethyl substituted fatty acyl group.

2. The compound of claim 1 wherein said compound is represented by the formula:
CH₃(CH₂)ₙ—CH(OR)—CH₂—CH(OR′)—(CH₂)₄—CH(CH₃)—CH(OH)—CH₃,
CH₃(CH₂)ₙ—CH(OR)—CH₂—CH(OR′)—(CH₂)₄—CH(CH₃)—CH(OH)—CH₂—CH₃,
CH₃(CH₂)ₙ—CH(OR)—CH₂—CH(OR′)—(CH₂)₄—CH(CH₃)—CH(OCH₃)—CH₃,
CH₃(CH₂)ₙ—CH(OR)—CH₂—CH(OR′)—(CH₂)₄—CH(CH₃)—CH(OCH₃)—CH₂—CH₃,
CH₃(CH₂)ₙ—CH(OR)—CH₂—CH(OR′)—(CH₂)₄—CH(CH₃)—C(O)—CH₃, or
CH₃(CH₂)ₙ—CH(OR)—CH₂—CH(OR′)—(CH₂)₄—CH(CH₃)—C(O)—CH₂—CH₃.

3. The compound of claim 1 wherein n is an integer from 20 to 30.

4. The compound of claim 1 wherein said compound has a formula:
CH₃—(CH₂)ₙ—CH(OR)—CH₂—CH(OR′)—(CH₂)₄—CH(CH₃)—CH(OH)—R″
the unsaturated fatty acyl group is an polyunsaturated fatty acyl group; and
the polymethyl substituted fatty acyl group has a formula:
CH₃—(CH₂)ₘ—(CH₂—CH(CH₃))ₓ—C(O)—
wherein m is an integer from 8 to 22; and x is 3 or 4.

5. The compound of claim 4 wherein the polyunsaturated fatty acyl group is a polyunsaturated omega-3 fatty acyl group.

6. The compound of claim 4, wherein R″ is ethyl; n is an integer from 24 to 32; m is an integer from 9 to 14; and x is 4.

7. The compound of claim 4, wherein R″ is ethyl; n is an integer from 24 to 32; m is an integer from 12 to 17; and x is 3.

8. The compound of claim 1 wherein R″ is methyl or ethyl.

9. The compound of claim 4, wherein m is an integer from 9 to 14; and x is 4.

10. The compound of claim 4, wherein m is an integer from 12 to 17; and x is 3.

11. The compound of claim 1, wherein the unsaturated fatty acyl group comprises a polyunsaturated fatty acyl group having 16 to 22 carbon atoms.

12. The compound of claim 1, wherein the unsaturated fatty acyl group comprises an omega-3 fatty acyl group represented by a formula:
CH₃—CH₂—CH═CH—(CH₂)ₐ—CH═CH—(CH₂)_{b}—CH═CH—CH₂—C(O)—
wherein a and b are integers and a+b=6 or a+b=8.

13. The compound of claim 1, wherein Y is —H; Z is —OH; n is an integer from 20 to 30; the unsaturated fatty acyl group comprises a 16-carbon polyunsaturated fatty acyl group or an 18 carbon polyunsaturated acyl group; and the polymethyl substituted fatty acyl group has a formula:
CH₃—(CH₂)ₘ—(CH₂—CH(CH₃))ₓ—C(O)—
wherein m is an integer from 9 to 17; and x is 3 or 4.

14. The compound of claim 1, wherein R″ is methyl or ethyl; the unsaturated fatty acyl group is an omega-3 polyunsaturated fatty acyl group having 16 carbon atoms; and the polymethyl substituted fatty acyl group is a C₂₅-mycocerosyl group.

15. A composition for disrupting biofilm produced by Mycobacterium, wherein the composition comprises an effective amount of the compound of claim 1.

16. A method for disrupting biofilm produced by Mycobacterium comprising contacting the biofilm with a composition comprising an effective amount of the compound of claim 1.

17. A method for aiding dispersal of biofilm produced by Mycobacterium comprising contacting the biofilm with a composition comprising an effective amount of the compound of claim 1.

18. A method for inhibiting formation of biofilm produced by Mycobacterium on a surface, comprising contacting the surface with a composition comprising an effective amount of the compound of claim 1.

19. A method for treating Mycobacterium infection in a subject in need thereof, comprising administering to the subject an effective amount of a composition comprising the compound of claim 1.

20. A pharmaceutical composition for treating a Mycobacterium infection comprising an effective amount of the compound of claim 1 and a pharmaceutically acceptable carrier.
